(19) United States
(12) Patent Application Publication
(10) Pub. No.: US 2018/0222953 A1
(43) Pub. Date: Aug. 9, 2018

(54) SARCOMERE BIOSENSOR AND METHODS OF USE THEREOF

(71) Applicant: REGENTS OF THE UNIVERSITY OF MINNESOTA, Minneapolis, MN (US)

(72) Inventors: Joseph M. Metzger, Minneapolis, MN (US); Anthony Vetter, Minneapolis, MN (US)

(21) Appl. No.: 15/881,478

(22) Filed: Jan. 26, 2018

Related U.S. Application Data
(60) Provisional application No. 62/451,539, filed on Jan. 27, 2017.

(51) Int. Cl. C07K 14/47; C09K 11/06; A01K 67/027; G01N 33/68; G01N 33/50; G01N 21/64

(52) U.S. Cl. CPC C07K 14/4716; C09K 11/06; A01K 67/0275; G01N 33/6887; G01N 33/502; G01N 2021/6439; G01N 21/6428; C07K 2319/60; C09K 2211/14; A01K 2217/05; A01K 2227/105; G01N 33/5061

(57) ABSTRACT

Certain embodiments of the invention provide a polypeptide comprising:
(a) an amino acid sequence encoding a first chromophore;
(b) a first linker;
(c) an amino acid sequence having at least about 80% sequence identity to an amino acid sequence encoding a troponin C;
(d) a second linker; and
(e) an amino acid sequence encoding a second chromophore;
wherein the first and second chromophores are a Förster resonance energy transfer (FRET) donor-acceptor pair; wherein the first chromophore is a donor chromophore and the second chromophore is an acceptor chromophore, or wherein the first chromophore is an acceptor chromophore and the second chromophore is a donor chromophore; and wherein the polypeptide is capable of integrating into a sarcomere. Certain embodiments of the invention also provide methods of using such polypeptides.

### RELATED APPLICATION

This application claims the benefit of priority of U.S. Provisional Application Ser. No. 62/451,539 filed on Jan. 27, 2017, which application is incorporated by reference herein.

### GOVERNMENT FUNDING

This invention was made with government support under R01 HL59301-17 awarded by the National Institutes of Health. The government has certain rights in the invention.

### BACKGROUND OF THE INVENTION

Excellent tools have been in place for decades to track muscle membrane excitation and the intracellular Ca²⁺ transient. Sarcomere function, however, has been a “black box” in terms of being able to assess, in real time, the activation of the myofilaments in live intact myocytes. Specifically, experiments investigating myocyte function have been typically conducted using steady state conditions so findings must be extrapolated to the intact myocyte under physiological conditions. The inability to directly monitor sarcomere activation in live cardiac myocytes has been a major limitation that has hampered progress in the field.

Accordingly, new compositions and methods are needed to monitor sarcomere activation in live cells.

### SUMMARY OF THE INVENTION

Thus, certain embodiments of the invention provide a polypeptide comprising:
(a) an amino acid sequence encoding a first chromophore;
(b) a first linker;
(c) an amino acid sequence having at least about 80% sequence identity to an amino acid sequence encoding a troponin C;
(d) a second linker; and
(e) an amino acid sequence encoding a second chromophore;

wherein the first and second chromophores are a Förster resonance energy transfer (FRET) donor-acceptor pair; wherein the first chromophore is a donor chromophore and the second chromophore is an acceptor chromophore, or wherein the first chromophore is an acceptor chromophore and the second chromophore is a donor chromophore; and wherein the polypeptide is capable of integrating into a sarcomere.

Certain embodiments of the invention provide a polypeptide comprising, in order:
(a) an amino acid sequence encoding Clover;
(b) a first linker;
(c) an amino acid sequence encoding human TNNC1;
(d) a second linker; and
(e) an amino acid sequence encoding mRuby2;

wherein the polypeptide is capable of integrating into a sarcomere.

Certain embodiments of the invention provide a nucleic acid encoding a polypeptide described herein.

Certain embodiments of the invention provide an expression cassette comprising a nucleic acid described herein.

Certain embodiments of the invention provide a vector comprising an expression cassette described herein.

Certain embodiments of the invention provide a cell comprising a polypeptide described herein, a nucleic acid described herein, an expression cassette described herein or a vector described herein.

Certain embodiments of the invention provide a non-human animal comprising a cell described herein.

Certain embodiments of the invention provide a transgenic non-human animal whose genome comprises a DNA transgene, which encodes a polypeptide described herein.

Certain embodiments of the invention provide a cell or cell line derived from a non-human animal described herein.

Certain embodiments of the invention provide a method for detecting structural changes in troponin C in a living cell, comprising 1) measuring in a FRET assay the fluorescent emission from a living cell expressing a polypeptide as described herein; and 2) detecting structural changes in troponin C when changes in the fluorescent emission are detected.

Certain embodiments of the invention provide a method for detecting sarcomere activation in a living cell, comprising 1) measuring in a FRET assay the fluorescent emission from a living cell expressing a polypeptide described herein; and 2) detecting sarcomere activation when changes in the fluorescent emission are detected.

Certain embodiments of the invention provide a method for screening biologically active agents that inhibit or promote sarcomere activation, comprising 1) contacting a living cell expressing a polypeptide as described herein with a test biologically active agent; 2) measuring the fluorescent emission from the cell in a FRET assay; and 3) detecting an effect of the test agent on sarcomere activation and thereby identifying an agent that inhibits or promotes sarcomere activation.

Certain embodiments of the invention provide a method for screening sarcomere protein mutations that inhibit or promote sarcomere activation, comprising 1) measuring the fluorescent emission from a living cell in a FRET assay, wherein the living cell expresses a polypeptide as described herein and comprises one or more mutations in a sarcomere protein; and 2) detecting an effect of the one or more mutations on sarcomere activation and thereby identifying a mutation that inhibits or promotes sarcomere activation.

### BRIEF DESCRIPTION OF THE FIGURES

FIG. 1. Schematic of cTnC_{sarcometer}. Here, the donor, a GFP variant, Clover, is fused to the N-terminus of human cTnC joined via a 20 amino acid flexible linker. The acceptor RFP variant, mRuby2, is fused to the C-terminus via an 8 amino acid flexible linker.

FIG. 2. Replacement efficiency of transgene. Transgene replacement is expressed as a percent of total cardiac troponin C as determined by densitometry using LiCor Odyssey after western blot. Blots were probed with anti-cTnC specific primary antibodies. Values mean±SEM, n=3.

FIG. 3. Nucleic acid sequence for cTnC_{sarcometer} (Example 1).

FIGS. 4A-E. Expression and function of the cardiac myocyte incorporated Sarcometer Biosensor. FIG. 4A, Model of the cardiac sarcomere. FIG. 4B, Western blot of cTnC expression in isolated myocytes reveals stoichiometric replacement of endogenous cTnC by the higher molecular weight Sarcometer cTnC. FIG. 4C, Immunofluorescence with confocal microscopy showing localization of biosensor is restricted between α-actinin delineated z-discs, magnified detail in insets. Scale bar is 10 μm. FIG. 4D, Representative single cardiac myocyte trace of simultaneous sarcomere length, Clover, and mRuby2 dynamics measured at 37° C. FIG. 4E, Amplitude normalized Clover (light grey) and sarcomere length (black) dynamics in single cell from (FIG. 4D) reveals a biosensor conformational change preceding sarcomere length change.

FIGS. 5A-D. Effects of β-Adrenergic Stimulation on the Sarcometer signal. FIG. 5A, Ensemble average traces of absolute SL shortening (left) and amplitude normalized SL shortening (right) in isolated myocytes before and after treatment with 50 nM isoproterenol (n=34 pairs). FIG. 5B, Summary statistics from sarcomere dynamics show increased shortening amplitude and faster kinetics upon treatment with isoproterenol (n=34 pairs). FIG. 5C, Ensemble average traces of absolute Clover intensity signal (left) and amplitude normalized Clover signal (right) in isolated myocytes before and after treatment with 50 nM isoproterenol (n=24 pairs). FIG. 5D, Summary statistics from fluorescence dynamics reveal an increased peak Clover fluorescence intensity and faster kinetics upon treatment with isoproterenol (n=24 pairs). Cells are measured at 30° C. with 0.2 Hz stimulation; FIGS. 5A,C, ΔSL bar represents a 5% change from baseline, ΔClover bar represents a 1% change from baseline. FIGS. 5B,D, Mean±S.E.M. are presented, Paired two-tailed t-test: *P<0.05.

FIGS. 6A-D. Thin filament targeted small molecules probe Sarcometer function in live cardiac myocyte. FIG. 6A, Ensemble average traces of SL shortening (left) in isolated myocytes before and after treatment with 10 μM W7 and summary statistics (right) from sarcomere dynamics reveal decreased contractile amplitude (n=8 pairs). FIG. 6B, Ensemble average traces of Clover transient (left) in isolated myocytes before and after treatment with 10 μM W7 and summary statistics (right) of Clover signal reveal decreased peak fluorescence intensity (n=8 pairs). FIG. 6C, Ensemble average traces of SL shortening (left) in isolated myocytes before and after treatment with 10 μM Pimobendan with summary statistics (right) from sarcomere dynamics showing increased contractile amplitude (n=11 pairs). FIG. 6D, Ensemble average traces of Clover signal (left) in isolated myocytes before and after treatment with 10 μM Pimobendan and summary statistics (right) of Clover intensity changes reveal increased peak fluorescence intensity (n=11 pairs). Myocytes measured at 37° C. with 0.2 Hz stimulation; ΔSL bar represents a 5% change from baseline, ΔClover bar represents a 1% change from baseline. Mean±S.E.M are presented, Paired two-tailed t-test: *P<0.05.

FIG. 7A-D. Myosin targeted small molecules probe thin-filament activation in a live cardiac myocyte. FIG. 7A, Ensemble average traces of SL shortening (left) in isolated myocytes before and after treatment with 250 nM Omecamtiv mecarbil and summary statistics (right) from sarcomere dynamics reveal increased contractile amplitude (n=14 pairs). FIG. 7B, Ensemble average traces of Clover signal (left) in isolated myocytes before and after treatment with 250 nM Omecamtiv mecarbil and summary statistics (right) of Clover intensity changes reveal no difference in peak fluorescence intensity (P=0.2847) (n=17 pairs). FIG. 7C, Ensemble average traces of SL shortening (left) in isolated myocytes before and after treatment with 10 μM para-Nitroblebbistatin and summary statistics (right) from sarcomere dynamics reveal decreased contractile amplitude (n=8 pairs). FIG. 7D, Ensemble average traces of Clover signal (left) in isolated myocytes before and after treatment with 10 μM para-Nitroblebbistatin and summary statistics (right) of Clover intensity changes reveal no difference in peak fluorescence intensity (P=0.8930) (n=8 pairs). Myocytes are measured at 37° C. with 0.2 Hz stimulation; ΔSL bar represents a 5% change from baseline, ΔClover bar represents a 1% change from baseline. Mean±S.E.M are presented, Paired two-tailed t-test: P<0.05.

FIG. 8. Model of Sarcometer Biosensor incorporated into the sarcomere. The myofilament incorporated Sarcometer biosensor stoichiometrically replaced endogenous troponin C. The Sarcometer is a fusion protein of the fluorescent protein Clover and mRuby2 to the N- and C-termini of cTnC by 20 and 8 amino acid flexible linkers respectively. The conformational changes that occur upon activation cause FRET as seen by anti-correlated signals in the Clover and mRuby2 fluorescence.

FIG. 9. Modified IonOptix Experimental Setup and Light Path. Here 505 nm and >695 nm light is reflected off a 505 nm dichroic long pass filter in the microscope turret before travelling to dichroic mirror 1 (650 nm) which permits the >695 nm transmitted light to the CCD. Light shorter than 650 nm is reflected to dichroic mirror 2 (565 nm) where the light is split between the two PMTs. Bandpass filters are used with Band-pass Filter 1 being a hard-coated 592/43 nm filter and Band-pass Filter 2 being a hard-coated 534/20 nm filter.

FIG. 10. Isolated myocytes from transgenic Sarcometer Biosensor mice have normal contractility when compared to cardiac myocyte results from Ntg littermates. There is no significant (N.S.) difference between any of the measured contractile parameters of the isolated myocytes of transgenic mice (n=24) and myocytes from their non-transgenic littermates (n=20). Mean±S.E.M. are presented, unpaired t-test.

FIGS. 11A-B. Gross heart anatomical measurements in Sarcometer biosensor mice. FIG. 11A, There is no significant (N.S.) difference between the heart weight, body weight, or tibia length of transgenic mice (n=6) and their non-transgenic littermates (n=6).

FIG. 11B, H&E staining of transverse cardiac sections showed no patho-histological changes. Scale bar represents 0.5 mm. Mean±S.E.M. are presented, unpaired t-test.

FIG. 12. Two-dimensional M-Mode Echocardiography of Sarcometer Biosensor Mice. Normal left ventricular diameters in systole and diastole as well as fractional shortening for both transgenic mice (n=14) and their non-transgenic littermates (n=4) as measured by M-Mode echocardiography. Mean±S.E.M. are presented, unpaired t-test. N.S.=not significant.

FIGS. 13A-C. Temperature dependence of contractility and thin-filament activation. Isolated myocytes show temperature dependence to the kinetics of contractility and thin filament activation as measured by sarcomere length and Clover intensity changes. Times represented are all from the electrical stimulus. FIG. 13A, Myocytes are paced at 0.2 Hz and measured at 23° C. (n=9 for SL and n=12 for Clover), FIG. 13B, Myocytes are paced at 0.2 Hz and measured at 30° C. (n=37 for SL and n=27 for Clover), FIG. 13C, Myocytes are paced at 0.2 Hz and measured at 37° C. (n=32 for SL and n=32 for Clover). Mean±S.E.M. are presented, unpaired t-test *P<0.05.

FIGS. 14A-B. Rat cardiac myocytes transduced using Sarcometer recombinant adenovirus does not alter myocyte contractile function. FIG. 14A, Representative single myocyte trace of simultaneous Clover, mRuby2, and sarcomere length recording. FIG. 14B, Summary statistics from the averaged transients reveals no changes in SL amplitude or kinetics (n=5). Mean±S.E.M. are presented, two-tailed unpaired t-test. N.S.=not significant.

FIGS. 15A-B. W7 has no effect on cardiac myocyte calcium transients. FIG. 15A, Ensemble average single myocyte transients incubated in Fura-2 AM before and after treatment with 10 μM W7. FIG. 15B, Summary statistics from the averaged transients reveals no changes in calcium amplitude or kinetics (n=4 pairs). Mean±S.E.M. are presented, two-tailed paired t-test. N.S.=not significant.

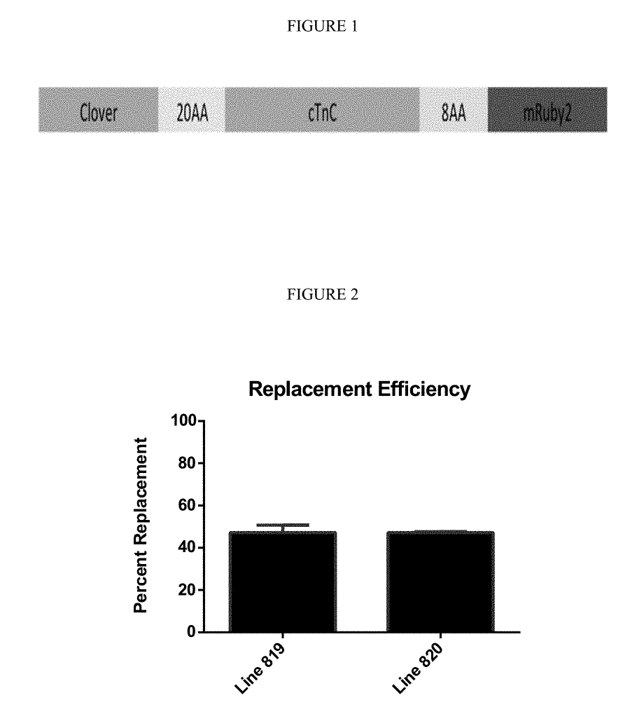

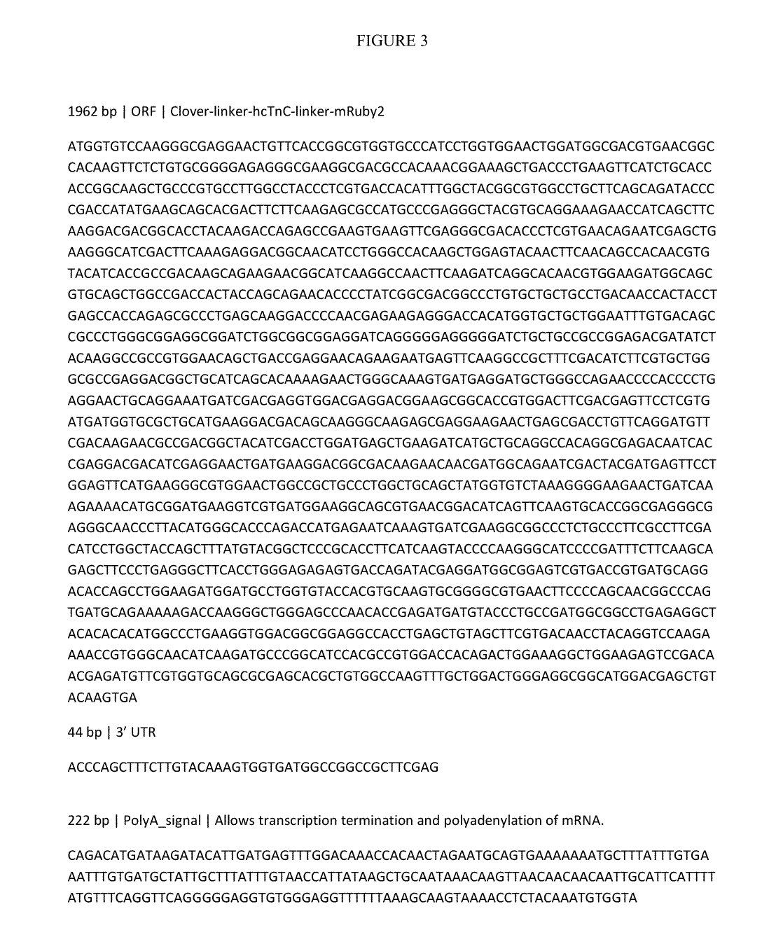

### DETAILED DESCRIPTION

As described herein, a platform has been developed that features a rationally designed biosensor, which may be used for beat-to-beat analysis (cardiac muscle activation) of living adult cardiac myocyte function. Additionally, as described in the Example, a transgenic mouse harboring this biosensor in the heart has also been developed.

Accordingly, certain embodiments of the invention provide a polypeptide comprising:
(a) an amino acid sequence encoding a first chromophore;
(b) a first linker;
(c) an amino acid sequence having at least 80% sequence identity to an amino acid sequence encoding a troponin C;
(d) a second linker; and
(e) an amino acid sequence encoding a second chromophore;

wherein the first and second chromophores are a Förster resonance energy transfer (FRET) donor-acceptor pair; wherein the first chromophore is a donor chromophore and the second chromophore is an acceptor chromophore, or wherein the first chromophore is an acceptor chromophore and the second chromophore is a donor chromophore; and wherein the polypeptide is capable of integrating into a sarcomere. Such a polypeptide may be used in a FRET assay to obtain real-time information on proximity changes between the chromophores resulting from structural changes in troponin C.

As used herein, the term “sarcomere” refers to the basic functional unit of a striated muscle, comprising actin, myosin, tropomyosin and the heterotrimeric troponin complex; held in tight stoichiometry capable of responding to calcium to coordinate muscle contraction.

As used herein, the term “capable of integrating into a sarcomere” means having the capability to replace the endogenous protein and incorporate into the sarcomere while maintaining the normal protein stoichiometry (i.e. not overexpression).

In certain embodiments, the polypeptide comprises elements (a)-(e) in order, either as a-b-c-d-e or e-d-c-b-a. In certain embodiments, the elements (a)-(e) are operably linked to each other (e.g., through a peptide bond). For example, in certain embodiments, the first chromophore is at the N′terminus of the polypeptide and is operably linked at its C-terminus to a first linker; the C-terminus of the first linker is operably linked to the N′terminus of the amino acid sequence having at least 80% sequence identity to an amino acid sequence encoding troponin C; a second linker is operably linked to the C-terminus of the amino acid sequence having at least 80% sequence identity to an amino acid sequence encoding troponin C; and the C′terminus of the second linker is operably linked to the N′terminus of the second chromophore.

In certain embodiments of the invention, the polypeptide consists of, in order,
(a) an amino acid sequence encoding a first chromophore;
(b) a first linker;
(c) an amino acid sequence having at least 80% sequence identity to an amino acid sequence encoding a troponin C;
(d) a second linker; and
(e) an amino acid sequence encoding a second chromophore;

wherein the first and second chromophores are a FRET donor-acceptor pair; wherein the first chromophore is a donor chromophore and the second chromophore is an acceptor chromophore, or wherein the first chromophore is an acceptor chromophore and the second chromophore is a donor chromophore; and wherein the polypeptide is capable of integrating into a sarcomere.

Described in the Example is a particular embodiment of a polypeptide of the invention. This polypeptide embodiment comprises, in order (i.e., N′terminal to C′terminal),
(a) an amino acid sequence encoding Clover;
(b) a first linker;
(c) an amino acid sequence encoding human cardiac troponin C;
(d) a second linker; and
(e) an amino acid sequence encoding mRuby2;

wherein the polypeptide is capable of integrating into a sarcomere. This polypeptide has the following sequence:

(SEQ ID NO: 1)
MVSKGEELFTGVVPILVELDGDVNGHKFSVRGEGEGDATNGKLTLK
FICTTGKLPVPWPTLVTTFGYGVACFSRYPDHMKQHDFFKSAMPEG
YVQERTISFKDDGTYKTRAEVKFEGDTLVNRIELKGIDFKEDGNIL
GHKLEYNFNSHNVYITADKQKNGIKANFKIRHNVEDGSVQLADHYQ
QNTPIGDGPVLLPDNHYLSHQSALSKDPNEKRDHMVLLEFVTAALG
GGGSGGGGSGGGGSAAAGDDIYKAAVEQLTEEQKNEFKAAFDIFVL
GAEDGCISTKELGKVMRMLGQNPTPEELQEMIDEVDEDGSGTVDFD
EFLVMMVRCMKDDSKGKSEEELSDLFRMFDKNADGYIDLDELKIML
QATGETITEDDIEELMKDGDKNNDGRIDYDEFLEFMKGVELAAALA
AAMVSKGEELIKENMRMKVVMEGSVNGHQFKCTGEGEGNPYMGTQT
MRIKVIEGGPLPFAFDILATSFMYGSRTFIKYPKGIPDFFKQSFPE
GFTWERVTRYEDGGVVTVMQDTSLEDGCLVYHVQVRGVNFPSNGPV
MQKKTKGWEPNTEMMYPADGGLRGYTHMALKVDGGGHLSCSFVTTY
RSKKTVGNIKMPGIHAVDHRLERLEESDNEMFVVQREHAVAKFAGL
GGGMDELYK.

Within this sequence, the amino acid sequence encoding Clover corresponds to amino acids 1-228; the first linker corresponds to amino acids 229-248; the amino acid sequence encoding human cardiac troponin C corresponds to amino acids 249-408; the second linker corresponds to amino acids 409-416; and the amino acid sequence encoding mRuby2 corresponds to amino acids 417-653.

Accordingly, certain embodiments of the invention provide a polypeptide comprising an amino acid sequence having at least about 75% sequence identity to SEQ ID NO:1. Certain embodiments of the invention provide a polypeptide comprising an amino acid sequence having at least about 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NO:1. Certain embodiments of the invention provide a polypeptide comprising an amino acid sequence having at least about 85% sequence identity to SEQ ID NO:1. Certain embodiments of the invention provide a polypeptide comprising an amino acid sequence having at least about 90% sequence identity to SEQ ID NO:1. Certain embodiments of the invention provide a polypeptide comprising an amino acid sequence having at least about 95% sequence identity to SEQ ID NO:1. Certain embodiments of the invention provide a polypeptide comprising an amino acid sequence having at least about 99% sequence identity to SEQ ID NO:1. Certain embodiments of the invention provide a polypeptide comprising SEQ ID NO:1.

Certain embodiments of the invention provide a polypeptide consisting of an amino acid sequence having at least about 75% sequence identity to SEQ ID NO:1. Certain embodiments of the invention provide a polypeptide consisting of an amino acid sequence having at least about 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NO:1.

Certain embodiments of the invention provide a polypeptide consisting of an amino acid sequence having at least about 85% sequence identity to SEQ ID NO:1. Certain embodiments of the invention provide a polypeptide consisting of an amino acid sequence having at least about 90% sequence identity to SEQ ID NO:1. Certain embodiments of the invention provide a polypeptide consisting of an amino acid sequence having at least about 95% sequence identity to SEQ ID NO:1. Certain embodiments of the invention provide a polypeptide consisting of an amino acid sequence having at least about 99% sequence identity to SEQ ID NO:1. Certain embodiments of the invention provide a polypeptide consisting of SEQ ID NO:1.

Certain embodiments of the invention provide a polypeptide as described herein.

### Chromophores

As used herein, the term “chromophore” refers to an atom or group whose presence is responsible for the color of a molecule.

As described herein, the first and second chromophores are a Förster resonance energy transfer (FRET) donor-acceptor pair. FRET is a mechanism describing energy transfer between two light-sensitive molecules (e.g., chromophores, such as fluorophores). A donor molecule, initially in its electronic excited state, may transfer energy to an acceptor molecule through nonradiative dipole-dipole coupling. The transfer of energy leads to a reduction in the donor's fluorescence intensity and excited state lifetime, and an increase in the acceptor's emission intensity. The efficiency of this energy transfer is inversely proportional to the sixth power of the distance between donor and acceptor, making FRET extremely sensitive to small changes in distance. A pair of molecules that interact in such a manner that FRET occurs is generally referred to as a donor-acceptor pair.

It is within the skill of the art to select donor-acceptor pairs and any combination of chromophores that can function as a donor-acceptor pair in a FRET assay may be used in the invention. For example, the skilled artisan may consider selecting a high quantum yield donor, a high absorbing acceptor and/or chromophores with significant overlap in their spectra.

In certain embodiments, the first chromophore is a donor chromophore and the second chromophore is an acceptor chromophore.

In certain embodiments, the first chromophore is an acceptor chromophore and the second chromophore is a donor chromophore.

In certain embodiments, the chromophore is a fluorophore. In certain embodiments, the fluorophore is capable of detection in vivo. Non-limiting examples of possible fluorophores include: Aquamarine, mCerulean, mTurquoise, mTurquoise2, CyPet, SCFP3A, Amber, mVenus, Ypet, SYFP2, SYFP2A, Clover, LSSmOrgange, mRuby2, ECFP, CFP, YFP, GFP, EGFP, Citrine, EYFP, mCherry and DsRed.

Accordingly, in certain embodiments, the donor and/or acceptor chromophore is selected from the group consisting of Aquamarine, mCerulean, mTurquoise, mTurquoise2, CyPet, SCFP3A, Amber, mVenus, Ypet, SYFP2, SYFP2A, Clover, LSSmOrgange, mRuby2, ECFP, CFP, YFP, GFP, EGFP, Citrine (e.g., mCitrine), EYFP, mCherry and DsRed.

In certain embodiments, the first chromophore is Clover. In certain embodiments, the second chromophore is Clover. In certain embodiments, the amino acid sequence encoding the first or second chromophore has least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to:

(SEQ ID NO: 2)
MVSKGEELFTGVVPILVELDGDVNGHKFSVRGEGEGDATNGKLTLK
FICTTGKLPVPWPTLVTTFGYGVACFSRYPDHMKQHDFFKSAMPEG
YVQERTISFKDDGTYKTRAEVKFEGDTLVNRIELKGIDFKEDGNIL
GHKLEYNFNSHNVYITADKQKNGIKANFKIRHNVEDGSVQLADHYQ
QNTPIGDGPVLLPDNHYLSHQSALSKDPNEKRDHMVLLEFVTAA.

In certain embodiments, the first chromophore is mRuby2. In certain embodiments, the second chromophore is mRuby2. In certain embodiments, the amino acid sequence encoding the first or second chromophore has least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to:

(SEQ ID NO: 3)
MVSKGEELIKENIVIRMKVVMEGSVNGHQFKCTGEGEGNPYMGTQT
MRIKVIEGGPLPFAFDILATSFMYGSRTFIKYPKGIPDFFKQSFPE
GFTWERVTRYEDGGVVTVMQDTSLEDGCLVYHVQVRGVNFPSNGPV
MQKKTKGWEPNTEMMYPADGGLRGYTHMALKVDGGGHLSCSFVTTY
RSKKTVGNIKMPGIHAVDHRLERLEESDNEMFVVQREHAVAKFAGL
GGGMDELYK.

Non-limiting examples of possible FRET donor-acceptor pairs include Clover-mRuby2, mCerulean-mVenus, mCerulean-Amber, mCerulean-SYFP2A, mTurquoise-mVenus, CFP-YFP, CFP-GFP, ECFP-EGFP, ECFP-mVenus, mCerulean-EYFP, mCerulean-mCitrine, ECFP-mCherry, ECFP-DsRed, EGFP-EYFP.

In certain embodiments, the first chromophore is Clover and the second chromophore is mRuby2. In certain embodiments, the first chromophore is mRuby2 and the second chromophore is Clover.

### Linkers

As described herein, the first and second linkers connect the first chromophore to the N′terminus of troponin C and the C′terminus of troponin C to the second chromophore, respectively. These linkers may comprise any combination of amino acids, provided 1) that the linker does not inhibit the native function of troponin C; and 2) the polypeptide of the invention is capable of functioning in a FRET assay. As used herein, the term, “does not inhibit the native function of troponin C” means that the activity of a sarcomere comprising a polypeptide of the invention is comparable to the activity of a sarcomere in a corresponding wildtype cell (e.g., has at least about 95%, 99% or 100% of the activity of a sarcomere in a corresponding wildtype cell). The amino acid sequence of the linker may vary depending on the selection of the chromophores and the troponin C sequence. However, the linkers are typically short and are, e.g., about 5 to about 30 amino acids in length, or about 5 to about 25 amino acids in length, or about 8 to about 20 amino acids in length.

In certain embodiments, the first linker is about 18 to about 22 amino acids in length. In certain embodiments, the first linker is about 19 to about 21 amino acids in length. In certain embodiments, the first linker is about 20 amino acids in length.

In certain embodiments, the amino acid sequence encoding the first linker is enriched in leucine, glycine, serine, and/or alanine residues. As used herein, the term “enriched” refers to an amino acid sequence consisting of at least about 60%, 65%, 70%, 75%, 80%, 85%, 90%, 95%, 99% or 100% of the specified amino acid residue(s). In certain embodiments, the amino acid sequence encoding the first linker consists of at least about 85% leucine, glycine, serine, and/or alanine residues. In certain embodiments, the amino acid sequence encoding the first linker consists of at least about 95% leucine, glycine, serine, and/or alanine residues. In certain embodiments, the amino acid sequence encoding the first linker consists of leucine, glycine, serine, and/or alanine residues.

In certain embodiments, the first linker comprises an amino acid sequence having at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to LGGGGSGGGGSGGGGSAAAG (SEQ ID NO:4). In certain embodiments, the first linker comprises an amino acid sequence having at least about 85% sequence identity to SEQ ID NO:4. In certain embodiments, the first linker comprises an amino acid sequence having at least about 90% sequence identity to SEQ ID NO:4. In certain embodiments, the first linker comprises an amino acid sequence having at least about 95% sequence identity to SEQ ID NO:4. In certain embodiments, the first linker comprises an amino acid sequence having at least about 99% sequence identity to SEQ ID NO:4. In certain embodiments, the first linker comprises SEQ ID NO:4.

In certain embodiments, the first linker consists of an amino acid sequence having at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to LGGGGSGGGGSGGGGSAAAG (SEQ ID NO:4). In certain embodiments, the first linker consists of an amino acid sequence having at least about 85% sequence identity to SEQ ID NO:4. In certain embodiments, the first linker consists an amino acid sequence having at least about 90% sequence identity to SEQ ID NO:4. In certain embodiments, the first linker consists of an amino acid sequence having at least about 95% sequence identity to SEQ ID NO:4. In certain embodiments, the first linker consists of an amino acid sequence having at least about 99% sequence identity to SEQ ID NO:4. In certain embodiments, the first linker consists of SEQ ID NO:4.

In certain embodiments, the second linker is about 6 to about 10 amino acids in length. In certain embodiments, the second linker is about 7 to about 9 amino acids in length. In certain embodiments, the second linker is about 8 amino acids in length.

In certain embodiments, the amino acid sequence encoding the second linker is enriched in leucine and/or alanine residues. In certain embodiments, the amino acid sequence encoding the second linker consists of at least about 85% leucine and/or alanine residues. In certain embodiments, the amino acid sequence encoding the second linker consists of at least about 95% leucine and/or alanine residues. In certain embodiments, the amino acid sequence encoding the second linker consists of leucine and/or alanine residues.

In certain embodiments, the second linker comprises an amino acid sequence having at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to LAAALAAA (SEQ ID NO:5). In certain embodiments, the second linker comprises an amino acid sequence having at least about 85% sequence identity to SEQ ID NO:4. In certain embodiments, the second linker comprises an amino acid sequence having at least about 90% sequence identity to SEQ ID NO:5. In certain embodiments, the second linker comprises an amino acid sequence having at least about 95% sequence identity to SEQ ID NO:5. In certain embodiments, the second linker comprises an amino acid sequence having at least about 99% sequence identity to SEQ ID NO:5. In certain embodiments, the second linker comprises SEQ ID NO:5.

In certain embodiments, the second linker consists of an amino acid sequence having at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to LAAALAAA (SEQ ID NO:5). In certain embodiments, the second linker consists of an amino acid sequence having at least about 85% sequence identity to SEQ ID NO:5. In certain embodiments, the second linker consists an amino acid sequence having at least about 90% sequence identity to SEQ ID NO:5. In certain embodiments, the second linker consists of an amino acid sequence having at least about 95% sequence identity to SEQ ID NO:5. In certain embodiments, the second linker consists of an amino acid sequence having at least about 99% sequence identity to SEQ ID NO:5. In certain embodiments, the second linker consists of SEQ ID NO:5.

### Troponin C

Troponin C is a part of the troponin complex. It contains four calcium-binding EF hands, although different isoforms may have fewer than four functional calcium-binding subdomains. It is a component of thin filaments (along with actin and tropomyosin). It contains an N lobe and a C lobe. The C lobe serves a structural purpose and binds to the N domain of troponin I (TnI). The C lobe can bind either Ca²⁺ or Mg²⁺. The N lobe, which binds only Ca²⁺, is the regulatory lobe and binds to the C domain of troponin I after calcium binding. There are tissue specific subtypes of troponin C. For example, in humans, there are two tissue specific isoforms: 1) slow troponin C, TNNC1 (also known as, troponin C, slow-twitch skeletal muscle; troponin C, cardiac; TNC; or cTnC; 3p21.3-p14.3; OMIM 191040; UniProtKB—P63316); and 2) troponin C, Fast TNNC2 (also known as troponin C, fast skeletal; 20q12-q13.11; OMIM 191039; UniProKB—P02585). Troponin C proteins are also expressed in other mammalian species, for example, in mouse (UniProKB—P19123 (Tnnc1); P20801 (Tnnc2)), rat (UniProKB—Q4PP99 (Tnnc1); Q304F3 (Tnnc2)), rabbit (UniProKB—P02591 (TNNC1); P02586 (TNNC2)), pig (UniProKB—P63317 (TNNC1); P02587 (TNNC2)) chicken (UniProKB—P09860 (TNNC1); P02588 (TNNC2)), bovine (UniProKB—P63315 (TNNC1); Q148C2 (TNNC2)), zebrafish (UniProKB—Q800V7 (tnnc1a); Q61Q64 (tnnclb); Q918U8 (tnnc2)), crayfish (UniProKB—P06707 (Troponin C, isotype alpha); P06708 (Troponin C, isotype gamma)), drosophila (UniProKB—P47947 (Troponin C, isoform 1); P47948 (Troponin C, isoform 2); P47949 (Troponin C, isoform 3)) and Rhesus macaque (UniProKB—G7MV95 (TNNC1); F7HGA7 (TNNC2)).

As described herein, a polypeptide of the invention comprises an amino acid sequence having at least 80% sequence identity to an amino acid sequence encoding a troponin C. In certain embodiments, a polypeptide of the invention comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99% or 100% sequence identity an amino acid sequence encoding a troponin C. In certain embodiments, a polypeptide of the invention comprises an amino acid sequence having at least 85% sequence identity to an amino acid sequence encoding a troponin C. In certain embodiments, a polypeptide of the invention comprises an amino acid sequence having at least 90% sequence identity to an amino acid sequence encoding a troponin C. In certain embodiments, a polypeptide of the invention comprises an amino acid sequence having at least 95% sequence identity to an amino acid sequence encoding a troponin C. In certain embodiments, a polypeptide of the invention comprises an amino acid sequence having at least 99% sequence identity to an amino acid sequence encoding a troponin C. In certain embodiments, a polypeptide of the invention comprises an amino acid sequence having 100% sequence identity to an amino acid sequence encoding a troponin C.

As described herein, one skilled in the art may modify an amino acid sequence encoding a troponin C by inserting amino acids, deleting amino acids or introducing point mutations, provided: 1) the modifications do not inhibit the native function of troponin C; and 2) the polypeptide of the invention is capable of functioning in a FRET assay. For example, the starting methionine may be removed from a TNNC peptide sequence, as it is being incorporated into a fusion protein (i.e., a polypeptide of the invention).

In certain embodiments, the troponin C is slow troponin C (TNNC1).

In certain embodiments, the troponin C is troponin C, Fast (TNNC2).

In certain embodiments, the troponin C is a mammalian troponin C. In certain embodiments, the troponin C is a human troponin C.

In certain embodiments, the troponin C is human TNNC1 (i.e., human cTnC). In certain embodiments, a polypeptide of the invention comprises an amino acid sequence having at least 80% sequence identity to: DDIYKAAVEQLTEEQKNEFKAAFDIFVLGAEDGCISTKELGKVMRMLGQNPTPEELQEM IDEVDEDGSGTVDFDEFLVMMVRCMKDD SKGKSEEELSDLFRMFDKNADGYIDLDELK IMLQATGETITEDDIEELMKDGDKNNDGRIDYDEFLEFMKGVE (SEQ ID NO:6). In certain embodiments, a polypeptide of the invention comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NO:6. In certain embodiments, a polypeptide of the invention comprises an amino acid sequence having at least 85% sequence identity to SEQ ID NO:6. In certain embodiments, a polypeptide of the invention comprises an amino acid sequence having at least 90% sequence identity to SEQ ID NO:6. In certain embodiments, a polypeptide of the invention comprises an amino acid sequence having at least 95% sequence identity to SEQ ID NO:6. In certain embodiments, a polypeptide of the invention comprises an amino acid sequence having at least 99% sequence identity to SEQ ID NO:6. In certain embodiments, a polypeptide of the invention comprises an amino acid sequence having at least 100% sequence identity to SEQ ID NO:6.

### Nucleic Acids, Expression Cassettes, Vectors, Cells and Animals

Certain embodiments of the invention provide a nucleic acid encoding a polypeptide described herein. In certain embodiments, the nucleic acid further comprises a promoter.

Certain embodiments of the invention provide an expression cassette comprising a nucleic acid sequence described herein and a promoter operably linked to the nucleic acid.

In certain embodiments, the promoter may be any promoter as described herein.

In certain embodiments, the promoter is a regulatable promoter. Nucleic acid sequences that are under the control of regulatable promoters are expressed only or to a greater or lesser degree in the presence of an inducing or repressing agent, (e.g., transcription under control of the metallothionein promoter is greatly increased in presence of certain metal ions). Regulatable promoters include responsive elements (REs) that stimulate transcription when their inducing factors are bound. For example, there are REs for serum factors, steroid hormones, retinoic acid, cyclic AMP, and tetracycline and doxycycline. Promoters containing a particular RE can be chosen in order to obtain a regulatable response and in some cases, the RE itself may be attached to a different promoter, thereby conferring regulatability to the encoded nucleic acid sequence. Thus, by selecting the appropriate promoter (constitutive versus regulatable; strong versus weak), it is possible to control both the existence and level of expression of a nucleic acid sequence in the genetically modified cell. If the nucleic acid sequence is under the control of an regulatable promoter, delivery of the therapeutic agent in situ is triggered by exposing the genetically modified cell in situ to conditions for permitting transcription of the nucleic acid sequence, e.g., by intraperitoneal injection of specific inducers of the regulatable promoters which control transcription of the agent. For example, in situ expression of a nucleic acid sequence under the control of the metallothionein promoter in genetically modified cells is enhanced by contacting the genetically modified cells with a solution containing the appropriate (i.e., inducing) metal ions in situ.

In certain embodiments, the promoter is a constitutive promoter. Naturally-occurring constitutive promoters control the expression of essential cell functions. As a result, a nucleic acid sequence under the control of a constitutive promoter is expressed under all conditions of cell growth. Constitutive promoters include the promoters for the following genes which encode certain constitutive or “housekeeping” functions: hypoxanthine phosphoribosyl transferase (HPRT), dihydrofolate reductase (DHFR), adenosine deaminase, phosphoglycerol kinase (PGK), pyruvate kinase, phosphoglycerol mutase, the beta-actin promoter, and other constitutive promoters known to those of skill in the art. In addition, many viral promoters function constitutively in eukaryotic cells. These include: the early and late promoters of SV40; the long terminal repeats (LTRs) of Moloney Leukemia Virus and other retroviruses; and the thymidine kinase promoter of Herpes Simplex Virus, among many others.

In certain embodiments, the promoter is a selective promoter designed to limit the expression of the polypeptide to a particular cell type. In certain embodiments, the promoter is a selective promoter designed to limit the expression of the polypeptide to cardiac cells. For example, in certain embodiments, the promoter is an alpha myosin heavy chain (aMHC) promoter.

In certain embodiments, the expression cassette further comprises an expression control sequence (e.g., an enhancer) operably linked to the nucleic acid sequence. Expression control sequences and techniques for operably linking sequences together are well known in the art.

The nucleic acid material (e.g., an expression cassette encoding a polypeptide described herein) can be introduced into the cell in vitro, ex vivo or in vivo by genetic transfer methods, such as transfection, transduction or pronuclear injection, to provide a genetically modified cell. Various expression vectors (i.e., vehicles for facilitating delivery of exogenous nucleic acid into a target cell, such as a plasmid) are known to one of ordinary skill in the art.

Accordingly, certain embodiments of the invention provide a vector comprising an expression cassette described herein.

As used herein, “transfection of cells” refers to the acquisition by a cell of new nucleic acid material by incorporation of added DNA. Thus, transfection refers to the insertion of nucleic acid into a cell using physical or chemical methods. Several transfection techniques are known to those of ordinary skill in the art including calcium phosphate DNA co-precipitation, DEAE-dextran, electroporation, cationic liposome-mediated transfection, tungsten particle-facilitated microparticle bombardment, and strontium phosphate DNA co-precipitation.

In contrast, “transduction of cells” refers to the process of transferring nucleic acid into a cell using a DNA or RNA virus. A RNA virus (i.e., a retrovirus) for transferring a nucleic acid into a cell is referred to herein as a transducing chimeric retrovirus. Exogenous nucleic acid material contained within the retrovirus is incorporated into the genome of the transduced cell.

A cell that has been transduced with a chimeric DNA virus (e.g., an adenovirus carrying a nucleic acid encoding a polypeptide of the invention), will not have the exogenous nucleic acid material incorporated into its genome but will be capable of expressing the exogenous nucleic acid material that is retained extrachromosomally within the cell. Examples of viral vectors include adenoviral, lentiviral, adeno-associated viral (AAV), self-complementary AAV (scAAV), poliovirus, HSV, or murine Maloney-based viral vector.

In certain embodiments, the vector is a viral vector. In certain embodiments, the viral vector is an adenoviral, lentiviral, adeno-associated viral (AAV), self-complementary AAV (scAAV), poliovirus, HSV, or murine Maloney-based viral vector.

Certain embodiments of the invention provide a cell comprising a nucleic acid described herein, an expression cassette described herein, a vector described herein or a polypeptide described herein.

For example, certain embodiments of the invention provide a cell described herein (e.g., in the Example).

In certain embodiments, the cell is a cardiac cell. In certain embodiments, the cell is a mammalian cardiac cell, such as a mouse cardiac cell.

In certain embodiments, the cell is a mammalian cell. In certain embodiments, the cell is a mouse cell. In certain embodiments, the cell is a human cell. In certain embodiments, the cell is an induced pluripotent stem cell (iPS cell).

Certain embodiments of the invention provide a non-human animal comprising a cell described herein. In certain embodiments, the non-human animal is a mammal. In certain embodiments, the non-human animal is a mouse.

Certain embodiments of the invention provide a transgenic non-human animal whose genome comprises a DNA transgene, which encodes a polypeptide of the invention (e.g., SEQ ID NO:1).

In certain embodiments, the transgenic non-human animal is heterozygous for the transgene. In certain embodiments, the transgenic non-human animal is homozygous for the transgene.

In certain embodiments, the transgenic non-human animal is a mammal. In certain embodiments, the transgenic non-human animal is a mouse.

In certain embodiments, the transgene is operably linked to a tissue-specific promoter. Certain embodiments of the invention provide a non-human animal (e.g., a non-human transgenic animal) as described herein.

Certain embodiments of the invention provide a cell or cell line derived from a non-human animal (e.g., a non-human transgenic animal) described herein.

### Kits

The present invention further includes kits for detecting sarcomere activation, comprising a nucleic acid, vector or cell described herein and instructions for detecting sarcomere activation using a FRET assay. Such kits may optionally contain one or more of: packaging material, other reagents or laboratory plasticware for use in the FRET assay.

### Methods

As described herein, a polypeptide of the invention may be used for real-time detection of sarcomere activation in live cells (e.g., cardiac myocytes). Thus, a polypeptide described herein may be used as a research tool to probe the function of the sarcomere in health and disease, including, e.g., elucidating the roles of sarcomeric activating ligands, the physiological significance of myofilament mutations and post-translational modifications.

As described herein, FRET may be used to detect structural changes in a polypeptide of the invention. Specifically, FRET may be used to detect proximity changes between the first and second chromophores that result from structural changes in the troponin C protein, which occur during sarcomere activation. As used herein, the term “sarcomere activation” refers to when the regulatory elements of the myofilaments are turned on to enable contraction to proceed.

Accordingly, certain embodiments of the invention provide a method for detecting structural changes in troponin C in a living cell comprising 1) measuring the fluorescent emission from a living cell expressing a polypeptide described herein in a FRET assay; and 2) detecting structural changes in troponin C when changes in the fluorescent emission are detected. In certain embodiments, the assay is performed in vitro, in vivo or ex vivo.

In certain embodiments, the cell is a cardiac cell (e.g., a cardiac myocyte).

In certain embodiments, the cell is a skeletal muscle cell.

Certain embodiments of the invention provide a method for detecting sarcomere activation in a living cell comprising 1) measuring the fluorescent emission from a living cell expressing a polypeptide described herein in a FRET assay; and 2) detecting sarcomere activation when changes in the fluorescent emission are detected. In certain embodiments, such an assay may be used to assess the impact of varying physiological conditions on sarcomere activation/regulation, such as pacing, stress, load, and across species.

In certain embodiments, the assay is performed in vitro, in vivo or ex vivo.

In certain embodiments, the cell is a cardiac cell (e.g., a cardiac myocyte).

In certain embodiments, the cell is a skeletal muscle cell.

In certain embodiments, the method further comprises detecting sarcomere length (e.g., using a modified Ionoptix Calcium and Contractility system as described herein).

Certain embodiments of the invention provide a method for screening biologically active agents that inhibit or promote sarcomere activation, comprising 1) contacting a living cell expressing a polypeptide described herein with a test biologically active agent; 2) measuring the fluorescent emission from the cell in a FRET assay; and 3) detecting an effect of the test agent on sarcomere activation (e.g., as compared to a control, such as sarcomere activation in the cell prior to contact with the test agent or sarcomere activation in a cell not contacted with the test agent). In certain embodiments, the assay is performed in vitro, in vivo or ex vivo. For example, in embodiments where the method is performed in vivo, the cell may be contacted with the test agent by administering (e.g., systemic administration) the test agent to an animal comprising the cell (e.g., a transgenic animal).

In certain embodiments, the cell is a cardiac cell (e.g., a cardiac myocyte).

In certain embodiments, the cell is a skeletal muscle cell.

Certain embodiments of the invention provide a method for screening sarcomere protein mutations that inhibit or promote sarcomere activation, comprising 1) measuring the fluorescent emission from a living cell in a FRET assay, wherein the living cell expresses a polypeptide described herein and comprises one or more mutations in a sarcomere protein; and 2) detecting an effect of the one or more mutations on sarcomere activation (e.g., as compared to a control, such as sarcomere activation in a cell that expresses a polypeptide of the invention but does not comprise the one or more mutations).

As used herein, the term “sarcomere protein” refers to proteins that are part of the sarcomere, including, e.g., Telethonin (UniProKB—070549), Capz (UniProKB—P47753), Titin (UniProKB—A2ASS6), Myopalladin (UniProKB—Q86TC9), Nebulin (UniProKB—Q61953), Nebulette (UniProKB—Q0II04), cTnI (UniProKB—P48787), fsTnI (UniProKB—P13412), ssTnI (UniProKB—Q9WUZ5), cTnC/ssTnC (UniProKB—P63316), fsTnC (UniProKB—P02585), cTnT (UniProKB—P50752), fsTnT (UniProKB—Q9QZ47), ssTnT (UniProKB—088346), fsMyBP-C (UniProKB—Q5XKE0), ssMyBP-C (UniProKB—GOYZM8), cMyBP-C (UniProKB—070468), alpha Myosin Heavy Chain (UniProKB—Q02566), beta Myosin Heavy Chain (UniProKB—Q91Z83), Myosin Heavy Chain 7B (UniProKB—A2AQP0), MYL1 (fast skeletal LC) (UniProKB—P05977), MYL2 (ventricular RLC) (UniProKB—P51667), MYL3 (ventricular ELC) (UniProKB—P09542), MYL4 (atrial ELC) (UniProKB—P09541), MYL5 (fetal RLC) (UniProKB—Q02045), MYL6B (slow skeletal LC) (UniProKB—Q60605), MYL7 (atrial RLC) (UniProKB—Q5SVI8), MYL11 (fs phosphorylatable LC) (UniProKB—P97457), skeletal Actin (UniProKB—P68134), cardiac Actin (UniProKB—P68033), alpha Actinin 2 (UniProKB—Q91191), Obscurin (UniProKB—A2AAJ9), Ankyrin (UniProKB—Q02357), Tropomyosin 1 (UniProKB—P58771), Tropomyosin 2 (UniProKB—P58774), Tropomyosin 3 (UniProKB—P21107) and Tropomyosin 4 (UniProKB—Q6IRU2).

In certain embodiments, the assay is performed in vitro, in vivo or ex vivo.

In certain embodiments, the cell is a cardiac cell (e.g., a cardiac myocyte).

In certain embodiments, the cell is a skeletal muscle cell.

In certain embodiments, the cell is a mammalian cell, such as a mouse cell.

Certain embodiments of the invention provide a method as described herein.

### Certain Definitions

The term “nucleic acid” refers to deoxyribonucleotides or ribonucleotides and polymers thereof in either single- or double-stranded form, composed of monomers (nucleotides) containing a sugar, phosphate and a base which is either a purine or pyrimidine. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al. (1991) Nucl. Acids Res., 19:508; Ohtsuka et al. (1985) JBC, 260:2605; Rossolini et al. (1994) Mol. Cell. Probes, 8:91. A “nucleic acid fragment” is a fraction of a given nucleic acid molecule. Deoxyribonucleic acid (DNA) in the majority of organisms is the genetic material while ribonucleic acid (RNA) is involved in the transfer of information contained within DNA into proteins. The term “nucleotide sequence” refers to a polymer of DNA or RNA that can be single- or double-stranded, optionally containing synthetic, non-natural or altered nucleotide bases capable of incorporation into DNA or RNA polymers. The terms “nucleic acid,” “nucleic acid molecule,” “nucleic acid fragment,” “nucleic acid sequence or segment,” or “polynucleotide” may also be used interchangeably with gene, cDNA, DNA and RNA encoded by a gene.

By “portion” or “fragment,” as it relates to a nucleic acid molecule, sequence or segment of the invention, when it is linked to other sequences for expression, is meant a sequence having at least 80 nucleotides, more preferably at least 150 nucleotides, and still more preferably at least 400 nucleotides. If not employed for expressing, a “portion” or “fragment” means at least 9, preferably 12, more preferably 15, even more preferably at least 20, consecutive nucleotides, e.g., probes and primers (oligonucleotides), corresponding to the nucleotide sequence of the nucleic acid molecules of the invention.

The term “amino acid,” comprises the residues of the natural amino acids (e.g. Ala, Arg, Asn, Asp, Cys, Glu, Gln, Gly, His, Hyl, Hyp, Ile, Leu, Lys, Met, Phe, Pro, Ser, Thr, Trp, Tyr, and Val) in D or L form, as well as unnatural amino acids (e.g. phosphoserine, phosphothreonine, phosphotyrosine, hydroxyproline, gamma-carboxyglutamate; hippuric acid, octahydroindole-2-carboxylic acid, statine, 1,2,3,4,-tetrahydroisoquinoline-3-carboxylic acid, penicillamine, ornithine, citruline, α-methyl-alanine, para-benzoylphenylalanine, phenylglycine, propargylglycine, sarcosine, and tert-butylglycine). The term also comprises natural and unnatural amino acids bearing a conventional amino protecting group (e.g. acetyl or benzyloxycarbonyl), as well as natural and unnatural amino acids protected at the carboxy terminus (e.g. as a (C₁-C₆) alkyl, phenyl or benzyl ester or amide; or as an α-methylbenzyl amide). Other suitable amino and carboxy protecting groups are known to those skilled in the art (See for example, T. W. Greene, Protecting Groups In Organic Synthesis; Wiley: New York, 1981, and references cited therein).

The terms “protein,” “peptide” and “polypeptide” are used interchangeably herein. Polypeptide sequences specifically recited herein are written with the amino terminus on the left and the carboxy terminus on the right.

The invention encompasses isolated or substantially purified nucleic acid or protein compositions. In the context of the present invention, an “isolated” or “purified” DNA molecule or an “isolated” or “purified” polypeptide is a DNA molecule or polypeptide that exists apart from its native environment and is therefore not a product of nature. An isolated DNA molecule or polypeptide may exist in a purified form or may exist in a non-native environment such as, for example, a transgenic host cell. For example, an “isolated” or “purified” nucleic acid molecule or protein, or biologically active portion thereof, is substantially free of other cellular material, or culture medium when produced by recombinant techniques, or substantially free of chemical precursors or other chemicals when chemically synthesized. In one embodiment, an “isolated” nucleic acid is free of sequences that naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecule can contain less than about 5 kb, 4 kb, 3 kb, 2 kb, 1 kb, 0.5 kb, or 0.1 kb of nucleotide sequences that naturally flank the nucleic acid molecule in genomic DNA of the cell from which the nucleic acid is derived. A protein that is substantially free of cellular material includes preparations of protein or polypeptide having less than about 30%, 20%, 10%, 5%, (by dry weight) of contaminating protein. When the protein of the invention, or biologically active portion thereof, is recombinantly produced, preferably culture medium represents less than about 30%, 20%, 10%, or 5% (by dry weight) of chemical precursors or non-protein-of-interest chemicals. Fragments and variants of the disclosed nucleotide sequences and proteins or partial-length proteins encoded thereby are also encompassed by the present invention. By “fragment” or “portion” is meant a full length or less than full length of the nucleotide sequence encoding, or the amino acid sequence of, a polypeptide or protein.

“Naturally occurring” or “wildtype” is used to describe an object that can be found in nature as distinct from being artificially produced. For example, a protein or nucleotide sequence present in an organism (including a virus), which can be isolated from a source in nature and which has not been intentionally modified by man in the laboratory, is naturally occurring.

A “variant” of a molecule is a sequence that is substantially similar to the sequence of the native molecule. For nucleotide sequences, variants include those sequences that, because of the degeneracy of the genetic code, encode the identical amino acid sequence of the native protein. Naturally occurring allelic variants such as these can be identified with the use of well-known molecular biology techniques, as, for example, with polymerase chain reaction (PCR) and hybridization techniques. Variant nucleotide sequences also include synthetically derived nucleotide sequences, such as those generated, for example, by using site-directed mutagenesis that encode the native protein, as well as those that encode a polypeptide having amino acid substitutions. Generally, nucleotide sequence variants of the invention will have at least 40, 50, 60, to 70%, e.g., preferably 71%, 72%, 73%, 74%, 75%, 76%, 77%, 78%, to 79%, generally at least 80%, e.g., 81%-84%, at least 85%, e.g., 86%, 87%, 88%, 89%, 90%, 91%, 92%, 93%, 94%, 95%, 96%, 97%, to 98%, sequence identity to the native (endogenous) nucleotide sequence.

“Conservatively modified variations” of a particular nucleic acid sequence refers to those nucleic acid sequences that encode identical or essentially identical amino acid sequences, or where the nucleic acid sequence does not encode an amino acid sequence, to essentially identical sequences. Because of the degeneracy of the genetic code, a large number of functionally identical nucleic acids encode any given polypeptide. For instance the codons CGT, CGC, CGA, CGG, AGA, and AGG all encode the amino acid arginine. Thus, at every position where an arginine is specified by a codon, the codon can be altered to any of the corresponding codons described without altering the encoded protein. Such nucleic acid variations are “silent variations” which are one species of “conservatively modified variations.” Every nucleic acid sequence described herein which encodes a polypeptide also describes every possible silent variation, except where otherwise noted. One of skill will recognize that each codon in a nucleic acid (except ATG, which is ordinarily the only codon for methionine) can be modified to yield a functionally identical molecule by standard techniques. Accordingly, each “silent variation” of a nucleic acid which encodes a polypeptide is implicit in each described sequence.

“Recombinant DNA molecule” is a combination of DNA sequences that are joined together using recombinant DNA technology and procedures used to join together DNA sequences as described, for example, in Sambrook and Russell, Molecular Cloning: A Laboratory Manual, Cold Spring Harbor, N.Y.: Cold Spring Harbor Laboratory Press (3^{rd} edition, 2001).

The terms “heterologous DNA sequence,” “exogenous DNA segment” or “heterologous nucleic acid,” each refer to a sequence that originates from a source foreign to the particular host cell or, if from the same source, is modified from its original form. Thus, a heterologous gene in a host cell includes a gene that is endogenous to the particular host cell but has been modified. The terms also include non-naturally occurring multiple copies of a naturally occurring DNA sequence. Thus, the terms refer to a DNA segment that is foreign or heterologous to the cell, or homologous to the cell but in a position within the host cell nucleic acid in which the element is not ordinarily found. Exogenous DNA segments are expressed to yield exogenous polypeptides.

A “homologous” DNA sequence is a DNA sequence that is naturally associated with a host cell into which it is introduced.

“Wild-type” refers to the normal gene, or organism found in nature without any known mutation.

“Genome” refers to the complete genetic material of an organism.

The term “gene” is used broadly to refer to any segment of nucleic acid associated with a biological function. Genes include coding sequences and/or the regulatory sequences required for their expression. For example, gene refers to a nucleic acid fragment that expresses mRNA, functional RNA, or a specific protein, including its regulatory sequences. Genes also include nonexpressed DNA segments that, for example, form recognition sequences for other proteins. Genes can be obtained from a variety of sources, including cloning from a source of interest or synthesizing from known or predicted sequence information, and may include sequences designed to have desired parameters. In addition, a “gene” or a “recombinant gene” refers to a nucleic acid molecule comprising an open reading frame and including at least one exon and (optionally) an intron sequence. The term “intron” refers to a DNA sequence present in a given gene which is not translated into protein and is generally found between exons.

A “vector” is defined to include, inter alia, any viral vector, plasmid, cosmid, phage or binary vector in double or single stranded linear or circular form which may or may not be self transmissible or mobilizable, and which can transform prokaryotic or eukaryotic host either by integration into the cellular genome or exist extrachromosomally (e.g., autonomous replicating plasmid with an origin of replication).

“Cloning vectors” typically contain one or a small number of restriction endonuclease recognition sites at which foreign DNA sequences can be inserted in a determinable fashion without loss of essential biological function of the vector, as well as a marker gene that is suitable for use in the identification and selection of cells transformed with the cloning vector. Marker genes typically include genes that provide tetracycline resistance, hygromycin resistance or ampicillin resistance.

“Expression cassette” as used herein means a DNA sequence capable of directing expression of a particular nucleotide sequence in an appropriate host cell, comprising a promoter operably linked to the nucleotide sequence of interest which is operably linked to termination signals. It also typically comprises sequences required for proper translation of the nucleotide sequence. The coding region usually codes for a protein of interest but may also code for a functional RNA of interest, for example antisense RNA or a nontranslated RNA, in the sense or antisense direction. The expression cassette comprising the nucleotide sequence of interest may be chimeric, meaning that at least one of its components is heterologous with respect to at least one of its other components. The expression cassette may also be one that is naturally occurring but has been obtained in a recombinant form useful for heterologous expression. The expression of the nucleotide sequence in the expression cassette may be under the control of a constitutive promoter or of an inducible promoter that initiates transcription only when the host cell is exposed to some particular external stimulus. In the case of a multicellular organism, the promoter can also be specific to a particular tissue or organ or stage of development.

Such expression cassettes will comprise the transcriptional initiation region of the invention linked to a nucleotide sequence of interest. Such an expression cassette is provided with a plurality of restriction sites for insertion of the gene of interest to be under the transcriptional regulation of the regulatory regions. The expression cassette may additionally contain selectable marker genes.

The term “RNA transcript” refers to the product resulting from RNA polymerase catalyzed transcription of a DNA sequence. When the RNA transcript is a perfect complementary copy of the DNA sequence, it is referred to as the primary transcript or it may be a RNA sequence derived from posttranscriptional processing of the primary transcript and is referred to as the mature RNA. “Messenger RNA” (mRNA) refers to the RNA that is without introns and that can be translated into protein by the cell. “cDNA” refers to a single- or a double-stranded DNA that is complementary to and derived from mRNA.

“Regulatory sequences” and “suitable regulatory sequences” each refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences include enhancers, promoters, translation leader sequences, introns, and polyadenylation signal sequences. They include natural and synthetic sequences as well as sequences that may be a combination of synthetic and natural sequences. As is noted above, the term “suitable regulatory sequences” is not limited to promoters. However, some suitable regulatory sequences useful in the present invention will include, but are not limited to constitutive promoters, tissue-specific promoters, development-specific promoters, inducible promoters and viral promoters.

“5′ non-coding sequence” refers to a nucleotide sequence located 5′ (upstream) to the coding sequence. It is present in the fully processed mRNA upstream of the initiation codon and may affect processing of the primary transcript to mRNA, mRNA stability or translation efficiency (Turner et al. (1995) Mol. Biotech. 3:225).

“3′ non-coding sequence” refers to nucleotide sequences located 3′ (downstream) to a coding sequence and include polyadenylation signal sequences and other sequences encoding regulatory signals capable of affecting mRNA processing or gene expression. The polyadenylation signal is usually characterized by affecting the addition of polyadenylic acid tracts to the 3′ end of the mRNA precursor.

The term “translation leader sequence” refers to that DNA sequence portion of a gene between the promoter and coding sequence that is transcribed into RNA and is present in the fully processed mRNA upstream (5′) of the translation start codon. The translation leader sequence may affect processing of the primary transcript to mRNA, mRNA stability or translation efficiency.

The term “mature” protein refers to a post-translationally processed polypeptide without its signal peptide. “Precursor” protein refers to the primary product of translation of an mRNA. “Signal peptide” refers to the amino terminal extension of a polypeptide, which is translated in conjunction with the polypeptide forming a precursor peptide and which is required for its entrance into the secretory pathway. The term “signal sequence” refers to a nucleotide sequence that encodes the signal peptide.

“Promoter” refers to a nucleotide sequence, usually upstream (5′) to its coding sequence, which controls the expression of the coding sequence by providing the recognition for RNA polymerase and other factors required for proper transcription. “Promoter” includes a minimal promoter that is a short DNA sequence comprised of a TATA-box and other sequences that serve to specify the site of transcription initiation, to which regulatory elements are added for control of expression. “Promoter” also refers to a nucleotide sequence that includes a minimal promoter plus regulatory elements that is capable of controlling the expression of a coding sequence or functional RNA. This type of promoter sequence consists of proximal and more distal upstream elements, the latter elements often referred to as enhancers. Accordingly, an “enhancer” is a DNA sequence that can stimulate promoter activity and may be an innate element of the promoter or a heterologous element inserted to enhance the level or tissue specificity of a promoter. Promoters may be derived in their entirety from a native gene, or be composed of different elements derived from different promoters found in nature, or even be comprised of synthetic DNA segments. A promoter may also contain DNA sequences that are involved in the binding of protein factors that control the effectiveness of transcription initiation in response to physiological or developmental conditions.

The “initiation site” is the position surrounding the first nucleotide that is part of the transcribed sequence, which is also defined as position+1. With respect to this site all other sequences of the gene and its controlling regions are numbered. Downstream sequences (i.e. further protein encoding sequences in the 3′ direction) are denominated positive, while upstream sequences (mostly of the controlling regions in the 5′ direction) are denominated negative.

Promoter elements, particularly a TATA element, that are inactive or that have greatly reduced promoter activity in the absence of upstream activation are referred to as “minimal or core promoters.” In the presence of a suitable transcription factor, the minimal promoter functions to permit transcription. A “minimal or core promoter” thus consists only of all basal elements needed for transcription initiation, e.g., a TATA box and/or an initiator.

“Constitutive expression” refers to expression using a constitutive or regulated promoter. “Conditional” and “regulated expression” refer to expression controlled by a regulated promoter.

“Operably-linked” refers to the association of nucleic acid sequences on single nucleic acid fragment so that the function of one is affected by the other. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably-linked to regulatory sequences in sense or antisense orientation.

“Expression” refers to the transcription and/or translation in a cell of an endogenous gene, transgene, as well as the transcription and stable accumulation of sense (mRNA) or functional RNA. In the case of antisense constructs, expression may refer to the transcription of the antisense DNA only. Expression may also refer to the production of protein.

“Transcription stop fragment” refers to nucleotide sequences that contain one or more regulatory signals, such as polyadenylation signal sequences, capable of terminating transcription. Examples of transcription stop fragments are known to the art.

“Translation stop fragment” refers to nucleotide sequences that contain one or more regulatory signals, such as one or more termination codons in all three frames, capable of terminating translation. Insertion of a translation stop fragment adjacent to or near the initiation codon at the 5′ end of the coding sequence will result in no translation or improper translation. Excision of the translation stop fragment by site-specific recombination will leave a site-specific sequence in the coding sequence that does not interfere with proper translation using the initiation codon.

The terms “cis-acting sequence” and “cis-acting element” refer to DNA or RNA sequences whose functions require them to be on the same molecule.

The terms “trans-acting sequence” and “trans-acting element” refer to DNA or RNA sequences whose function does not require them to be on the same molecule.

“Homology” refers to the percent identity between two polynucleotides or two polypeptide sequences. Two DNA or polypeptide sequences are “homologous” to each other when the sequences exhibit at least about 75% to 85% (including 75%, 76%, 77%, 78%, 79%, 80%, 81%, 82%, 83%, 84%, and 85%), at least about 90%, or at least about 95% to 99% (including 95%, 96%, 97%, 98%, 99%) contiguous sequence identity over a defined length of the sequences.

The following terms are used to describe the sequence relationships between two or more sequences (e.g., nucleic acids, polynucleotides or polypeptides): (a) “reference sequence,” (b) “comparison window,” (c) “sequence identity,” (d) “percentage of sequence identity,” and (e) “substantial identity.”

(a) As used herein, “reference sequence” is a defined sequence used as a basis for sequence comparison. A reference sequence may be a subset or the entirety of a specified sequence; for example, as a segment of a full length cDNA, gene sequence or peptide sequence, or the complete cDNA, gene sequence or peptide sequence.

(b) As used herein, “comparison window” makes reference to a contiguous and specified segment of a sequence, wherein the sequence in the comparison window may comprise additions or deletions (i.e., gaps) compared to the reference sequence (which does not comprise additions or deletions) for optimal alignment of the two sequences. Generally, the comparison window is at least 20 contiguous nucleotides in length, and optionally can be 30, 40, 50, 100, or longer. Those of skill in the art understand that to avoid a high similarity to a reference sequence due to inclusion of gaps in the sequence a gap penalty is typically introduced and is subtracted from the number of matches.

Methods of alignment of sequences for comparison are well known in the art. Thus, the determination of percent identity between any two sequences can be accomplished using a mathematical algorithm. Non-limiting examples of such mathematical algorithms are the algorithm of Myers and Miller (1988) CABIOS, 4:11; the local homology algorithm of Smith et al. (1981) Adv. Appl. Math. 2:482; the homology alignment algorithm of Needleman and Wunsch, (1970) JMB, 48:443; the search-for-similarity-method of Pearson and Lipman, (1988) Proc. Natl. Acad. Sci. USA, 85:2444; the algorithm of Karlin and Altschul, (1990) Proc. Natl. Acad. Sci. USA, 87:2264, modified as in Karlin and Altschul, (1993) Proc. Natl. Acad. Sci. USA, 90:5873.

Computer implementations of these mathematical algorithms can be utilized for comparison of sequences to determine sequence identity. Such implementations include, but are not limited to: CLUSTAL in the PC/Gene program (available from Intelligenetics, Mountain View, Calif.); the ALIGN program (Version 2.0) and GAP, BESTFIT, BLAST, FASTA, and TFASTA in the Wisconsin Genetics Software Package, Version 8 (available from Genetics Computer Group (GCG), 575 Science Drive, Madison, Wis., USA). Alignments using these programs can be performed using the default parameters. The CLUSTAL program is well described by Higgins et al. (1988) Gene 73:237; Higgins et al. (1989) CABIOS 5:151; Corpet et al. (1988) Nucl. Acids Res. 16:10881; Huang et al. (1992) CABIOS 8:155; and Pearson et al. (1994) Meth. Mol. Biol. 24:307. The ALIGN program is based on the algorithm of Myers and Miller, supra. The BLAST programs of Altschul et al. (1990) JMB, 215:403; Nucl. Acids Res., 25:3389 (1990), are based on the algorithm of Karlin and Altschul supra.

Software for performing BLAST analyses is publicly available through the National Center for Biotechnology Information (available on the world wide web at ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached.

In addition to calculating percent sequence identity, the BLAST algorithm also performs a statistical analysis of the similarity between two sequences. One measure of similarity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleic acid sequence to the reference nucleic acid sequence is less than about 0.1, more preferably less than about 0.01, and most preferably less than about 0.001.

To obtain gapped alignments for comparison purposes, Gapped BLAST (in BLAST 2.0) can be utilized as described in Altschul et al. (1997) Nucleic Acids Res. 25:3389. Alternatively, PSI-BLAST (in BLAST 2.0) can be used to perform an iterated search that detects distant relationships between molecules. See Altschul et al., supra. When utilizing BLAST, Gapped BLAST, PSI-BLAST, the default parameters of the respective programs (e.g., BLASTN for nucleotide sequences, BLASTX for proteins) can be used. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix. See the world wide web at ncbi.nlm.nih.gov. Alignment may also be performed manually by visual inspection.

For purposes of the present invention, comparison of sequences for determination of percent sequence identity to another sequence may be made using the BlastN program (version 1.4.7 or later) with its default parameters or any equivalent program. By “equivalent program” is intended any sequence comparison program that, for any two sequences in question, generates an alignment having identical nucleotide or amino acid residue matches and an identical percent sequence identity when compared to the corresponding alignment generated by the preferred program.

(c) As used herein, “sequence identity” or “identity” in the context of two nucleic acid or polypeptide sequences makes reference to a specified percentage of residues in the two sequences that are the same when aligned for maximum correspondence over a specified comparison window, as measured by sequence comparison algorithms or by visual inspection. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity.” Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

(d) As used herein, “percentage of sequence identity” means the value determined by comparing two optimally aligned sequences over a comparison window, wherein the portion of the sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

(e)(i) The term “substantial identity” of sequences means that a polynucleotide comprises a sequence that has at least 70%, 71%, 72%, 73%, 74%, 75%, 76%, 77%, 78%, or 79%, at least 80%, 81%, 82%, 83%, 84%, 85%, 86%, 87%, 88%, or 89%, at least 90%, 91%, 92%, 93%, or 94%, and at least 95%, 96%, 97%, 98%, or 99% sequence identity, compared to a reference sequence using one of the alignment programs described using standard parameters. One of skill in the art will recognize that these values can be appropriately adjusted to determine corresponding identity of proteins encoded by two nucleotide sequences by taking into account codon degeneracy, amino acid similarity, reading frame positioning, and the like. Substantial identity of amino acid sequences for these purposes normally means sequence identity of at least 70%, at least 80%, 90%, at least 95%.

Another indication that nucleotide sequences are substantially identical is if two molecules hybridize to each other under stringent conditions (see below). Generally, stringent conditions are selected to be about 5° C. lower than the thermal melting point (Tₘ) for the specific sequence at a defined ionic strength and pH. However, stringent conditions encompass temperatures in the range of about 1° C. to about 20° C., depending upon the desired degree of stringency as otherwise qualified herein. Nucleic acids that do not hybridize to each other under stringent conditions are still substantially identical if the polypeptides they encode are substantially identical. This may occur, e.g., when a copy of a nucleic acid is created using the maximum codon degeneracy permitted by the genetic code. One indication that two nucleic acid sequences are substantially identical is when the polypeptide encoded by the first nucleic acid is immunologically cross reactive with the polypeptide encoded by the second nucleic acid.

(e)(ii) The term “substantial identity” in the context of a peptide indicates that a peptide comprises a sequence with at least 70%, 71%, 72%, 73%, 74%, 75%, 76%, 77%, 78%, or 79%, 80%, 81%, 82%, 83%, 84%, 85%, 86%, 87%, 88%, or 89%, at least 90%, 91%, 92%, 93%, or 94%, or 95%, 96%, 97%, 98% or 99%, sequence identity to the reference sequence over a specified comparison window. Optimal alignment is conducted using the homology alignment algorithm of Needleman and Wunsch, J. Mol. Biol. 48:443 (1970). An indication that two peptide sequences are substantially identical is that one peptide is immunologically reactive with antibodies raised against the second peptide. Thus, a peptide is substantially identical to a second peptide, for example, where the two peptides differ only by a conservative substitution.

For sequence comparison, typically one sequence acts as a reference sequence to which test sequences are compared. When using a sequence comparison algorithm, test and reference sequences are input into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

As noted above, another indication that two nucleic acid sequences are substantially identical is that the two molecules hybridize to each other under stringent conditions. The phrase “hybridizing specifically to” refers to the binding, duplexing, or hybridizing of a molecule only to a particular nucleotide sequence under stringent conditions when that sequence is present in a complex mixture (e.g., total cellular) DNA or RNA. “Bind(s) substantially” refers to complementary hybridization between a probe nucleic acid and a target nucleic acid and embraces minor mismatches that can be accommodated by reducing the stringency of the hybridization media to achieve the desired detection of the target nucleic acid sequence.

“Stringent hybridization conditions” and “stringent hybridization wash conditions” in the context of nucleic acid hybridization experiments such as Southern and Northern hybridizations are sequence dependent, and are different under different environmental parameters. Longer sequences hybridize specifically at higher temperatures. The thermal melting point (Tₘ) is the temperature (under defined ionic strength and pH) at which 50% of the target sequence hybridizes to a perfectly matched probe. Specificity is typically the function of post-hybridization washes, the critical factors being the ionic strength and temperature of the final wash solution. For DNA-DNA hybrids, the Tₘ can be approximated from the equation of Meinkoth and Wahl (1984) Anal. Biochem. 138:267; Tₘ 81.5° C.+16.6 (log M)+0.41 (% GC)−0.61 (% form)−500/L; where M is the molarity of monovalent cations, % GC is the percentage of guanosine and cytosine nucleotides in the DNA, % form is the percentage of formamide in the hybridization solution, and L is the length of the hybrid in base pairs. Tₘ is reduced by about 1° C. for each 1% of mismatching; thus, Tₘ, hybridization, and/or wash conditions can be adjusted to hybridize to sequences of the desired identity. For example, if sequences with >90% identity are sought, the Tₘ can be decreased 10° C. Generally, stringent conditions are selected to be about 5° C. lower than the Tₘ for the specific sequence and its complement at a defined ionic strength and pH. However, severely stringent conditions can utilize a hybridization and/or wash at 1, 2, 3, or 4° C. lower than the Tₘ; moderately stringent conditions can utilize a hybridization and/or wash at 6, 7, 8, 9, or 10° C. lower than the Tₘ; low stringency conditions can utilize a hybridization and/or wash at 11, 12, 13, 14, 15, or 20° C. lower than the Tₘ Using the equation, hybridization and wash compositions, and desired temperature, those of ordinary skill will understand that variations in the stringency of hybridization and/or wash solutions are inherently described. If the desired degree of mismatching results in a temperature of less than 45° C. (aqueous solution) or 32° C. (formamide solution), it is preferred to increase the SSC concentration so that a higher temperature can be used. An extensive guide to the hybridization of nucleic acids is found in Tijssen, Laboratory Techniques in Biochemistry and Molecular Biology Hybridization with Nucleic Acid Probes, part I chapter 2 “Overview of principles of hybridization and the strategy of nucleic acid probe assays” Elsevier, New York (1993). Generally, highly stringent hybridization and wash conditions are selected to be about 5° C. lower than the Tₘ for the specific sequence at a defined ionic strength and pH.

An example of highly stringent wash conditions is 0.15 M NaCl at 72° C. for about 15 minutes. An example of stringent wash conditions is a 0.2×SSC wash at 65° C. for 15 minutes (see, Sambrook, infra, for a description of SSC buffer). Often, a high stringency wash is preceded by a low stringency wash to remove background probe signal. An example medium stringency wash for a duplex of, e.g., more than 100 nucleotides, is 1×SSC at 45° C. for 15 minutes. An example low stringency wash for a duplex of, e.g., more than 100 nucleotides, is 4-6×SSC at 40° C. for 15 minutes. For short probes (e.g., about 10 to 50 nucleotides), stringent conditions typically involve salt concentrations of less than about 1.5 M, more preferably about 0.01 to 1.0 M, Na ion concentration (or other salts) at pH 7.0 to 8.3, and the temperature is typically at least about 30° C. and at least about 60° C. for long probes (e.g., >50 nucleotides). Stringent conditions may also be achieved with the addition of destabilizing agents such as formamide. In general, a signal to noise ratio of 2× (or higher) than that observed for an unrelated probe in the particular hybridization assay indicates detection of a specific hybridization. Nucleic acids that do not hybridize to each other under stringent conditions are still substantially identical if the proteins that they encode are substantially identical. This occurs, e.g., when a copy of a nucleic acid is created using the maximum codon degeneracy permitted by the genetic code.

Very stringent conditions are selected to be equal to the Tₘ for a particular probe. An example of stringent conditions for hybridization of complementary nucleic acids which have more than 100 complementary residues on a filter in a Southern or Northern blot is 50% formamide, e.g., hybridization in 50% formamide, 1 M NaCl, 1% SDS at 37° C., and a wash in 0.1×SSC at 60 to 65° C. Exemplary low stringency conditions include hybridization with a buffer solution of 30 to 35% formamide, 1M NaCl, 1% SDS (sodium dodecyl sulphate) at 37° C., and a wash in 1× to 2×SSC (20×SSC=3.0 M NaCl/0.3 M trisodium citrate) at 50 to 55° C. Exemplary moderate stringency conditions include hybridization in 40 to 45% formamide, 1.0 M NaCl, 1% SDS at 37° C., and a wash in 0.5× to 1×SSC at 55 to 60° C.

By “variant” polypeptide is intended a polypeptide derived from the native protein by deletion (so-called truncation) or addition of one or more amino acids to the N-terminal and/or C-terminal end of the native protein; deletion or addition of one or more amino acids at one or more sites in the native protein; or substitution of one or more amino acids at one or more sites in the native protein. Such variants may results form, for example, genetic polymorphism or from human manipulation. Methods for such manipulations are generally known in the art.

Thus, the polypeptides of the invention may be altered in various ways including amino acid substitutions, deletions, truncations, and insertions. Methods for such manipulations are generally known in the art. For example, amino acid sequence variants of the polypeptides can be prepared by mutations in the DNA. Methods for mutagenesis and nucleotide sequence alterations are well known in the art. See, for example, Kunkel (1985) Proc. Natl. Acad. Sci. USA 82:488; Kunkel et al. (1987) Meth. Enzymol. 154:367; U.S. Pat. No. 4,873,192; Walker and Gaastra (1983) Techniques in Mol. Biol. (MacMillan Publishing Co., and the references cited therein. Guidance as to appropriate amino acid substitutions that do not affect biological activity of the protein of interest may be found in the model of Dayhoff et al., Atlas of Protein Sequence and Structure (Natl. Biomed. Res. Found. 1978). Conservative substitutions, such as exchanging one amino acid with another having similar properties, are preferred.

Thus, the genes and nucleotide sequences of the invention include both the naturally occurring sequences as well as mutant forms. Likewise, the polypeptides of the invention encompass naturally occurring proteins as well as variations and modified forms thereof. Such variants will continue to possess the desired activity. In certain embodiments, the deletions, insertions, and substitutions of the polypeptide sequence encompassed herein may not produce radical changes in the characteristics of the polypeptide. However, when it is difficult to predict the exact effect of the substitution, deletion, or insertion in advance of doing so, one skilled in the art will appreciate that the effect will be evaluated by routine screening assays.

Individual substitutions deletions or additions that alter, add or delete a single amino acid or a small percentage of amino acids (typically less than 5%, more typically less than 1%) in an encoded sequence are “conservatively modified variations,” where the alterations result in the substitution of an amino acid with a chemically similar amino acid. Conservative substitution tables providing functionally similar amino acids are well known in the art. The following five groups each contain amino acids that are conservative substitutions for one another: Aliphatic: Glycine (G), Alanine (A), Valine (V), Leucine (L), Isoleucine (I); Aromatic: Phenylalanine (F), Tyrosine (Y), Tryptophan (W); Sulfur-containing: Methionine (M), Cysteine (C); Basic: Arginine (R), Lysine (K), Histidine (H); Acidic: Aspartic acid (D), Glutamic acid (E), Asparagine (N), Glutamine (Q). In addition, individual substitutions, deletions or additions which alter, add or delete a single amino acid or a small percentage of amino acids in an encoded sequence are also “conservatively modified variations.”

The term “transformation” refers to the transfer of a nucleic acid fragment into the genome of a host cell, resulting in genetically stable inheritance. Host cells containing the transformed nucleic acid fragments are referred to as “transgenic” cells, and organisms comprising transgenic cells are referred to as “transgenic organisms”.

“Transformed,” “transgenic,” “transduced” and “recombinant” refer to a host cell or organism into which a heterologous nucleic acid molecule has been introduced. The nucleic acid molecule can be stably integrated into the genome generally known in the art and are disclosed in Sambrook and Russell, supra. See also Innis et al., PCR Protocols, Academic Press (1995); and Gelfand, PCR Strategies, Academic Press (1995); and Innis and Gelfand, PCR Methods Manual, Academic Press (1999). Known methods of PCR include, but are not limited to, methods using paired primers, nested primers, single specific primers, degenerate primers, gene-specific primers, vector-specific primers, partially mismatched primers, and the like. For example, “transformed,” “transformant,” and “transgenic” cells have been through the transformation process and contain a foreign gene integrated into their chromosome. The term “untransformed” refers to normal cells that have not been through the transformation process.

“Genetically altered cells” denotes cells which have been modified by the introduction of recombinant or heterologous nucleic acids (e.g., one or more DNA constructs or their RNA counterparts) and further includes the progeny of such cells which retain part or all of such genetic modification.

As used herein, the term “derived” or “directed to” with respect to a nucleotide molecule means that the molecule has complementary sequence identity to a particular molecule of interest.

The term “therapeutically effective amount,” in reference to treating a disease state/condition, refers to an amount of vector either alone or as contained in a pharmaceutical composition that is capable of having any detectable, positive effect on any symptom, aspect, or characteristics of a disease state/condition when administered as a single dose or in multiple doses. Such effect need not be absolute to be beneficial.

The terms “treat” and “treatment” refer to both therapeutic treatment and prophylactic or preventative measures, wherein the object is to prevent or decrease an undesired physiological change or disorder. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

The invention will now be illustrated by the following non-limiting Examples.

### Example 1. Development, Validation and Implementation of a Novel Real Time Biosensor of Sarcomere Activation in Live Cardiac Muscle

The cardiac myocyte is elegantly designed for highly orchestrated changes in cytosolic [Ca²⁺] during excitation-contraction (EC) coupling (Bers, Med. Sci. Sports Exerc. 23, 1157 (1991); Bers, et al., Ann. N. Y. Acad. Sci. 853, 157 (1998); Bers, Nature 415, 198 (2002)). Impaired EC coupling is a prominent feature of the diseased and failing heart (Kranias and Dumas, J. Virol. 13, 146 (1974); Frank, et al., Basic Res. Cardiol. 97 Suppl 1, 172 (2002); MacLennan and Kranias, Nat. Rev. Mol. Cell Biol. 4, 566 (2003); Haghighi et al., J. Clin. Invest 111, 869 (2003); Schmitt et al., Science 299, 1410 (2003); Haghighi, et al., Biochem. Biophys. Res. Commun. 322, 1214 (2004); Kranias and Bers, Subcell. Biochem. 45, 523 (2007); Arvanitis et al., Eur. Heart J. 29, 2514 (2008); Haghighi et al., Hum. Mutat. 29, 640 (2008); Chen et al., FASEB J. 22, 1790 (2008)). The current experimental therapeutics pipeline for failing heart muscle largely focuses on modifications of the amplitude/kinetics of the intracellular Ca²⁺ transient that trigger the contractile event (Kranias and Bers, Subcell. Biochem. 45, 523 (2007); Kranias and Hajjar, Circ. Res. 110, 1646 (2012); Mattiazzi and Kranias, Heart Rhythm. 8, 784 (2011); Lorenz and Kranias, Am. J. Physiol 273, H2826 (1997); Koss and Kranias, Circ. Res. 79, 1059 (1996); Bers, Annu. Rev. Physiol 70, 23 (2008)). These approaches, while certainly worth pursuing, do raise several concerns. These include increasing SR Ca²⁺ load, which could have significant negative outcomes, such as arrhythmias and increased susceptibility to ischemic injury (Zhang et al., Circ. Res. 107, 800 (2010)). As heart disease is prominent in our country, new targets and alternative approaches are critically needed.

Additionally, in a wide range of inherited and acquired cardiac diseases, troponin dysfunction has been implicated to underlie the basis of excitation-contraction uncoupling (Davis et al., Physiol Rev. 88, 1567 (2008); Davis, et al., J. Mol. Cell Cardiol. 44, 891 (2008); Davis and Metzger, PLoS. One. 5, e9140 (2010); Michele, et al., Nat. Med. 5, 1413 (1999); Michele and Metzger, Trends Cardiovasc. Med. 10, 177 (2000); Michele and Metzger, J. Mol. Med. 78, 543 (2000); Arad, et al., Hum. Mol. Genet. 11, 2499 (2002); Fatkin et al., J. Clin. Invest 106, 1351 (2000); C. E. Seidman, J. G. Seidman, Basic Res. Cardiol. 93 Suppl 3, 13 (1998); J. G. Seidman, C. Seidman, Cell 104, 557 (2001)). Here, the severity of disease couples closely with the degree of sarcomere-based EC-uncoupling (Davis and Metzger, PLoS. One. 5, e9140 (2010); Ho et al., Circulation 102, 1950 (2000)). For example, in acute ischemia, hypoxia, and acidosis, the intracellular Ca²⁺ transient is normal and yet heart muscle performance is severely depressed. This is evidence of the central role sarcomere performance plays as the focal mediator of excitation-contraction uncoupling (Lee and Allen, J. Clin. Invest 88, 361 (1991)). Many have contributed to this knowledge base: J. Davis et al., Physiol Rev. 88, 1567 (2008); J. Davis, J. M. Metzger, PLoS. One. 5, e9140 (2010); J. Davis et al., Cell (2016); B. R. Thompson, E. M. Houang, Y. Y. Sham, J. M. Metzger, Biophys. J. 106, 2105 (2014); B. R. Thompson, J. M. Metzger, Anat. Rec. (Hoboken.) 297, 1663 (2014); R. J. Solaro, Circ. Res. 84, 122 (1999); J. Wattanapermpool, P. J. Reiser, R. J. Solaro, Am. J. Physiol 268, C323 (1995); R. J. Solaro, Adv. Exp. Med. Biol. 382, 109 (1995). However, one key challenge of these works is that they are typically conducted using steady state conditions so findings must be extrapolated to the intact myocyte under physiological conditions. This represents a key gap in understanding that is addressed here.

Described herein is a new tool for real-time detection of sarcomere activation in live cardiac myocytes. This permits the quantitative monitoring of myofilament activation in living adult cardiac myocytes. This innovation will permit unprecedented mechanistic insights into the function of the sarcomere in health and disease, including elucidating the roles of sarcomeric activating ligands and post-translation modifications that, heretofore, have not been possible to test in live myocytes.

Monitoring sarcomere activation in live cell recordings is a major unmet need and is a major impediment for the field to advance. Mechanistic studies of the sarcomere are essential to understand cardiac disease. This focus is required to advance new therapeutic approaches for the diseased heart. Directly assessing sarcomere function is critical because the sarcomere is the indispensable functional unit of the heart and breakdown in sarcomere function is central to the failing heart.

Described herein is the design and implementation of a new Live Cell Biosensor of myofilament activation; a breakthrough method for direct real-time monitoring of thin filament activation in living adult cardiac myocytes. Prior to this advance, it has not been possible to directly monitor myofilament activation in live myocytes. This is a major gap because many disease states, from acute ischemia to inherited cardiomyopathies, significantly uncouple intracellular Ca²⁺ from sarcomere activation (e.g., direct change in sarcomere function) (Davis et al., Physiol Rev. 88, 1567 (2008); Michele, et al., Nat. Med. 5, 1413 (1999); Lee and Allen, J. Clin. Invest 88, 361 (1991)). Thus, measurements of intracellular Ca²⁺ alone are not sufficient to gain full mechanistic insight into these disease states. Significance of this advance is seen by the measurements in living myocytes under physiological temperature and in conditions where excitation-contraction coupling and signaling processes are fully intact. This significant technological innovation will provide for unprecedented mechanistic insights into the function of the sarcomere in disease.

### Methods

### Gene Construct and Transgenesis

The 480 bp full length (less then starting methionine) human cardiac Troponin C (hcTnC) cDNA was synthesized de novo and engineered to be flanked on the 5′ end by a 20 amino acid flexible linker peptide enriched in leucine, glycine, serine, and alanine; and flanked on the 3′ end by an 8 amino acid flexible linker enriched in leucine and alanine. The 20 amino acid linker is inserted between a PasI restriction site at the 5′ end and an EcoRV restriction site at the 3′ end. The 8 amino acid linker is inserted between a PfoI restriction site at the 5′ end and a MI restriction site at the 3′ end. On the 5′ end of the 20 amino acid flexible linker is the 684 bp cDNA of the green fluorescent protein “Clover”. The Clover cDNA is inserted between a SalI restriction site at the 5′ end and the aforementioned PasI restriction site at the 3′ end. On the 3′ end of the 8 amino acid flexible linker is the 714 bp cDNA of the red fluorescent protein “mRuby2”. The mRuby2 cDNA is inserted between the aforementioned MI restriction site at the 5′ end and an FseI restriction site at the 3′ end. This Clover-hcTnC-mRuby2 gene construct will herein be referred to as “cTnC XFP”. When expressed in cardiac myocytes, the cTnC XFP gene product acts a FRET-based biosensor where conformational changes within the cTnC protein enhance fluorescence resonance energy transfer between the Clover protein and the mRuby2 protein. When excited at a peak excitation of 505 nm the Clover protein enhances the stimulated emission of the mRuby2 by FRET. This is shown schematically in FIG. 1. The cTnC XFP gene construct was inserted in a DNA vector downstream of an alpha myosin heavy chain (aMHC) promoter element for robust and cardiac specific expression in a mammalian system. This aMHC cTnC XFP vector was inserted by pronuclear injection into donor egg cells and implanted in C57/B16 murine surrogate mothers. Live pups were genotyped using the PCR sense primer located in mRuby2: 5′-CAACATCAAGATGCCCGGCA-3′ (SEQ ID NO:1), and the PCR antisense primer located in the SV40 PolyA region: 5′-CACTGCATTCTAGTTGTGGTTTGTCC-3′ (SEQ ID NO:2) with an annealing temperature of 62° C. Of the live pups, 5 founders carrying the transgene were identified and the line with greatest breeding vigor was selected for further validation.

### In Vivo Echocardiography

Eight to twenty week old NTG and cTnC XFP Tg mice were lightly anesthetized by 2.5% Isoflurane (Piramal Healthcare) and prepared for echocardiography. M-mode imaging was performed with a Vevo 2100 (Visual Sonics) at 1000 fps with the MS550D 22-55 MHz transducer. The parasternal short axis view was used to assess systolic and diastolic. M-mode imaging was also used to calculate fractional shortening (FS).

### Myocyte Isolation and Culture

Mice were injected with 0.25 cc 10,000U/ml heparin (McKesson cat#NDC 63739-964-25) and anesthetized 10 minutes later with 0.15 cc 50 mg/ml sodium pentobarbital (Vortech Pharmaceuticals). The heart was rapidly excised and placed in ice cold Krebs-Henseleit buffer (Sigma cat#K3753) modified with 25 mM HEPES (Sigma cat#H4034), 10 mM 2,3-Butanedione monoxime (Sigma cat#B0753), 30 mM Taurine (Sigma cat#T0625) and the aorta was cannulated. The mounted heart was placed on a perfusion system at 37° C. The heart was perfused with calcium free modified Krebs-Henseleit buffer with 2 mg/ml crudely purified collagenase for 10 minutes. The heart was then removed from the cannula, the atria were removed and the ventricles were dissected into pieces approximately 2 mm in diameter. The dissected ventricular tissue was then gently mechanically triturated in collagenase to liberate isolated myocytes. After brief low-speed centrifugation, isolated myocytes were resuspended in nominal calcium and 0.025 mg/ml bovine serum albumin (HyClone cat#SH30574.03) in a modified Kreb-Henseleit buffer. After the final trituration step, calcium was added back to the buffer step wise until reaching a final concentration of 2 mM CaCl₂. Myocytes were plated on laminin-coated (ThermoFisher cat#23017015) glass coverslips in M199 media (Gibco cat#31100-035) containing 5% fetal bovine serum (HyClone cat#SH30071.03) and incubated for one hour at 37° C. with 5% CO₂. After the one hour incubation, the media was changed to serum free M199 media and the myocytes were used for experiments within 5 hours.

### Real-Time Calcium, Contractility, and Biosensor Measurements

For real-time simultaneous sarcomere length and biosensor measurements, a modified Ionoptix Calcium and Contractility system was used. Sarcomere length changes were detected at 240 Hz sampling rate and fluorescence measurements were detected at 1000 Hz sampling rate with two photomultipler tubes. Measurements were made at 37° C. and a stimulation frequency of 0.2 Hz unless specified otherwise. For biosensor measurements, a Xenon arc lamp was used and filtered with a 500/10 nm hard-coated bandpass filter before entering the microscope turret. The microscope turret featured a 505 dichroic long pass filter. Fluorescent light from the sample was shunted through microscope side port where it was split by a dichroic mirror with light above 650 nm going towards the Myocam for sarcomere length detection and light below 650 nm being further shunted to an additional filter cube. In this final filter cube, a 565 nm dichroic long pass mirror split the light again. Light with wavelengths below 565 nm corresponding to the Clover protein was further cleaned up by a 534/20 nm hard-coated bandpass filter before reaching the photomultiplier tuber and light with wavelengths above 565 nm was further cleaned up by a 592/43 nm hard-coated bandpass filter before reaching the photomultiplier tube. For calcium fluorescence measurements, the mouse myocytes were pre-loaded for 20 minutes in M199 solution containing 1 μM Fura-2 AM (ThermoFisher Cat #F1221), and a ten minute period was allowed for deesterification. For calcium fluorescence measurements the 360/380 nm ratio was determined. For each myocyte, several contractions were measured, averaged, and analyzed using the IonWizard software.

### Immunofluorescence

Laminin coated coverslips containing isolated myocytes were treated with 4% paraformaldehyde in phosphate buffered saline for five minutes followed by two washes in phosphate buffered saline. Samples were blocked with 5% bovine serum albumin with 0.5% Triton X-100 in phosphate buffered saline for at least one hour. Primary antibody was applied at room temperature for one hour in phosphate buffered saline with 0.5% Triton X-100 and 0.5% BSA. Several washes were performed in PBS with 0.5% Triton X-100 and 5% BSA, followed by one hour incubation of the secondary antibody at room temperature in PBS with 0.5 Triton X-100 and 0.5% BSA. Coverslips were mounted on microscope slides with ProLong Diamond mounting medium (ThermoFisher Cat#P36965).

### Results

Live Cell Direct Detection of Sarcomere Activation—Design, Implementation and Validation.

As described herein, a unique system for real-time detection of sarcomere activation in live cardiac myocytes has been designed and validated. This innovation leads mechanistically-driven studies on the function of the sarcomere in health and disease, including elucidating the roles of sarcomeric activating ligands. Using this FRET-based sarcomere activation platform, the physiological significance of myofilament mutations and post-translational modifications, which heretofore have not been possible to test in live myocytes, may be determined.

Live Cell Sarcometer Development and Validation.

Förster resonance energy transfer (FRET) was implemented for dynamic real-time information on proximity changes between intra-molecular fluorescent probes engineered in cardiac troponin C (cTnC) (FIG. 1). FRET provides for live cell proximity changes between intra- or intermolecular fluorescent probes due to conformational changes in the proteins they are conjugated to (Davis, et al., J. Mol. Cell Cardiol. 44, 891 (2008); Davis and Metzger, PLoS. One. 5, e9140 (2010); Michele, et al., Nat. Med. 5, 1413 (1999); Michele and Metzger, Trends Cardiovasc. Med. 10, 177 (2000); Michele and Metzger, J. Mol. Med. 78, 543 (2000)). FRET based approaches have been previously utilized to characterize the stepwise movements of the troponin complex in vitro in response to calcium titration (Roy, et al., Nat. Methods 5, 507 (2008)). This information has been important in elucidating the nuances of the inter- and intramolecular movements of isolated myofilament proteins, however to date this technique has not been used to study myocyte contractility in living myocytes (Arad, et al., Hum. Mol. Genet. 11, 2499 (2002); Fatkin et al., J. Clin. Invest 106, 1351 (2000); C. E. Seidman, J. G. Seidman, Basic Res. Cardiol. 93 Suppl 3, 13 (1998); J. G. Seidman, C. Seidman, Cell 104, 557 (2001); Ho et al., Circulation 102, 1950 (2000); Lee and Allen, J. Clin. Invest 88, 361 (1991)). Technical limitations of traditional fluorescent dyes make them incompatible with live cells. However, now, through the use of genetically encoded fluorescent probes it is possible to introduce fluorophores through gene transfer/transgenesis to directly monitor myofilament activation in live myocytes. To our knowledge, this is the first time this technique has been development and validated for study of adult cardiac myocyte contractility in living myocytes.

A novel troponin C FRET-based biosensor construct was designed with discrete flexible linkers fusing fluorescent proteins Clover and mRuby2 engineered at the N- and C-terminal ends of cardiac troponin C (herein cTnC_{sarcometer})(FIG. 1). The cTnC_{sarcometer} serves as a FRET-based biosensor with Clover as the donor and mRuby2 as the acceptor. The experimental setup involves donor probe stimulation with blue light, hence Clover emits green light with a peak emission at 515 nm. A percentage of the energy required to excite the donor fluorophore is emitted as green light while another population of the photons undergoes intersystem crossing and excites the lower energy acceptor fluorophore “mRuby2”. Conformational changes that occur in cTnC upon binding to Ca²⁺ and other activating ligands (TnI/myosin) manifest as changes in fluorophore intensity. As the distance between the fluorophores decreases, the Clover intensity decreases while the mRuby2 intensity increases in an anti-correlated manner indicating FRET. Thus the Clover fluorescence is transferred through nonradiative processes to mRuby2 in a distance-dependent manner to then reflect real-time conformational changes occurring within the myofilament. Clover and mRuby2 are among the most sophisticated fluorescent proteins engineered to date, featuring excellent brightness, quantum yield, photostability and the highest Förster radius of any ratiometric FRET pair to date (Davis et al., Cell (2016)). Additionally, the capability of monitoring the red-shifted fluorescence enables detection outside of the range of the inherent autofluorescence of cardiac myocytes (Thompson, et al., Biophys. J. 106, 2105 (2014); Roy, et al., Nat. Methods 5, 507 (2008); Lam et al., Nat. Methods 9, 1005 (2012)). These attributes are all important to enable the first rationally designed biosensor capable of monitoring myofilament activation in live cardiac muscle cells.

### Discussion

This work has great potential for a sustained impact on the field. By illuminating the sarcomere “black box” in live cardiac muscle, fundamental mechanisms of cardiac contraction may be addressed, which was not possible with previous technologies. Decades old models of myofilament regulation will be challenged in this application, including establishing the key ligands of activation in live cells and testing the classic “blocked, closed, open” model of thin filament (Geeves, et al., Biochemistry 39, 9345 (2000); Geeves, S. S. Lehrer, Biophys. J. 107, 543 (2014); Geeves, K. C. Holmes, Adv. Protein Chem. 71, 161 (2005); Geeves, P. B. Conibear, Biophys. J. 68, 194S (1995); Lehrer, and Geeves, J. Muscle Res. Cell Mont. 35, 153 (2014); McKillop and Geeves, Biophys. J. 65, 693 (1993); Reiffert, et al., FEBS Lett. 384, 43 (1996); Schaertl, et al., Biochemistry 34, 15890 (1995); Gollapudi, et al., Biophys. J. 103, 940 (2012); Rao, et al., J. Biol. Chem. 286, 24135 (2011); Sia et al., J. Biol. Chem. 272, 18216 (1997); Swartz, et al., J. Mol. Biol. 361, 420 (2006)). Further, demonstrating myofilament inactivation as the basis of excitation-contraction uncoupling in live membrane intact cardiac myocyte is critical to dissecting the regulatory role of the sarcomere in health and disease.

Furthermore, innovation through development of a new FRET-based sarcomere activation sensor will drive discovery and mechanistic analysis of novel myofilament-acting molecules that may be developed to enhance heart performance in disease. Specifically, live cardiac muscle cell quantitative testing of novel small molecule modulators of live cell sarcomere activation will enable mechanistic insights into myofilament-based regulation of cardiac contractile function. This new approach further serves a long-term goal of targeting the sarcomere in advancing new treatments for acquired and inherited cardiac disorders.

### Example 2: Sarcomere Biosensor Elucidates Myofilament Activating Ligands in Live Cardiac Myocytes

The sarcomere is the functional unit of the heart. Sarcomere dysfunction has devastating consequences in both acquired and inherited cardiac diseases. However, it has not yet been possible to illuminate sarcomere performance in live cells. This is an important gap as cardiac muscle operates under highly dynamic conditions. Described herein is the design and implementation of a live cell reporter of sarcomere activation, which has been termed the Sarcometer. Under physiological conditions of intact excitation-contraction coupling the Sarcometer revealed the key regulatory functions of calcium and troponin to orchestrate the highly cooperative myofilament signal transduction process necessary to generate force. Unexpectedly, in contradistinction from long-standing theory, live cell data show that myosin binding is not required as an essential activating ligand for regulating the cardiac sarcomere. These results advance a paradigm shift in the mechanistic understanding of live-cell cardiac sarcomere regulation with implications for physiological beat-to-beat control of heart performance in health and disease.

### Introduction

The study described herein is focused on the cardiac sarcomere, the functional unit of the heart. The sarcomere is a near-liquid crystalline array of contractile and regulatory proteins exquisitely aligned in interdigitating myofilaments that govern beat-to-beat control of overall myocardial performance. Thin myofilament proteins that regulate thick myofilament myosin motor activity include actin, tropomyosin (Tm), and the troponin complex (Tn)(Gordon, et al. (2000) Physiol Rev. 80, 853-924; Farah, C. S. & Reinach, F. C. (1995) FASEB J. 9, 755-767). Troponin is a heterotrimeric regulatory complex comprised of the Tm binding subunit, troponin T (TnT), the calcium binding subunit, troponin C (TnC), and the inhibitory subunit, troponin I (TnI). Sarcomere-based regulation of contraction entails highly coordinated actions of the troponin complex, in intimate connection with Tm and actin, to direct muscle contraction. TnI, the molecular switch of the thin filament regulatory system, is anchored to actin at low [Ca²⁺] and switches binding to an exposed hydrophobic “sticky patch” of residues on TnC at high [Ca²⁺] to initiate contraction (Farah, et al. (1994) J. Biol. Chem. 269, 5230-5240; Sykes, B. D. (2003) Nat. Struct. Biol. 10, 588-589; Tobacman, L. S. (1996) Annu. Rev. Physiol 58, 447-481). This dramatic conformational change in TnC-TnI then orchestrates a long-range signal transduction cascade, mediated by Tm, to allow contraction. Thus, the sarcomere regulatory system is a highly integrative and cooperative signaling process in which the spatial alignment of Tm on actin is modeled to be finely controlled and tuned by Ca²⁺, troponin and myosin as interconnecting allosteric effectors (Gordon, et al. (2000) Physiol Rev. 80, 853-924).

Whereas excellent tools have been long available to quantitatively detect and monitor the electrical and intracellular Ca²⁺ handling properties of live cardiac muscle cells, real-time detection of sarcomere activation has been elusive in the physiological setting of the live cardiac myocyte. This gap has been a key roadblock to advancing knowledge as the sarcomere is the centerpiece of heart performance. This is particularly critical in that several prominent cardiac disorders are directly caused by defects in sarcomere structure and function (Seidman, C. (2002) Mayo Clin. Proc. 77, 1315-1319).

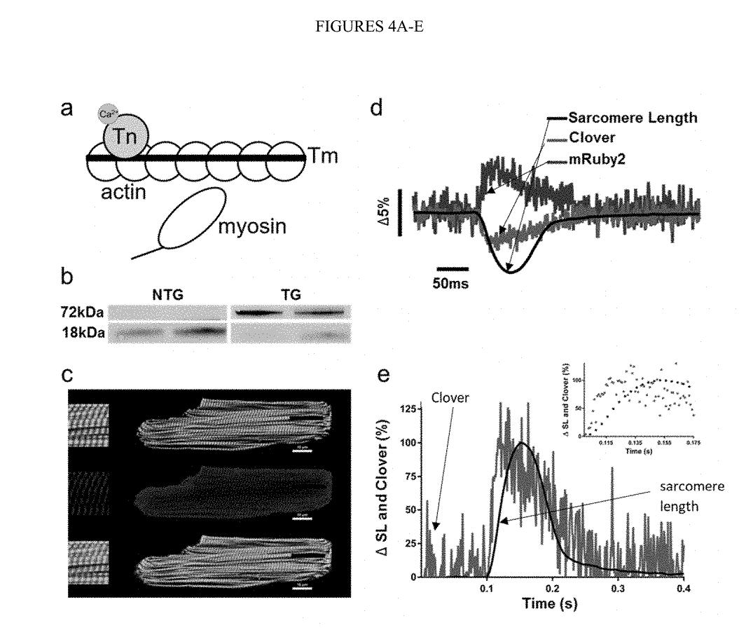

For decades, the prevailing model defining sarcomere regulation has centered on Ca²⁺, troponin and myosin as activating ligands, specifically serving as allosteric effectors orchestrating sequential block, closed and open state transitions in the myofilament regulatory system (Bremel, R. D. & Weber, A. (1972) Nat. New Biol. 238, 97-101; Hannon, et al. (1992) Circ. Res. 71, 984-991; McKillop, D. F. & Geeves, M. A. (1993) Biophys. J. 65, 693-701) (FIG. 4a). A wealth of evidence, derived primarily from isolated protein, electron microscopy, atomistic-based 3-D reconstructions and steady-state fiber reconstitution studies, has supported the notion that strong myosin binding is necessary to activate the sarcomere regulatory apparatus (Gordon, et al. (2000) Physiol Rev. 80, 853-924; Hannon, et al. (1992) Circ. Res. 71, 984-991; McKillop, D. F. & Geeves, M. A. (1993) Biophys. J. 65, 693-701; Geeves, M. A. & Holmes, K. C. (2005) Adv. Protein Chem. 71, 161-193). While informative, these findings are limited by non-physiological experimental conditions that are critical as cardiac muscle operates under highly dynamic excitation-contraction coupling control (Bers, D. M. (2002) Nature 415, 198-205). Prior to Applicant's invention described herein, it was not possible to determine the status of sarcomere activation and its regulation in the context of a living cardiac myocyte in real time.

Specifically, the development and implementation of an approach enabling the first investigations of sarcomeric activating ligands in the context of live cardiac myocytes is described herein. Using a myofilament biosensor, termed Sarcometer, in cardiac muscle cells under physiological conditions of live myocytes with intact excitation-contraction coupling and studied at physiological temperature, compelling new evidence of the fundamental ligands that enable cardiac sarcomere activation are provided. Specifically, the data shows that myosin binding, long thought to be the principal activating ligand for the sarcomere, notably requisite for the canonical “closed to open transition state” of the myofilaments to initiate contraction, has no effect on sarcomere activation when studied under the live cell conditions here. Data further show that full activation is evident by activating ligands Ca²⁺ and the troponin regulatory complex in frame with tropomyosin on the actin-based thin myofilament. These new findings provide new insights into the basic principles of cardiac muscle activation and should accelerate efforts toward directly targeting sarcomere performance in health and disease.

### Results

The Sarcometer Detects Physiological Activation of Thin Filament in Live Myocytes.

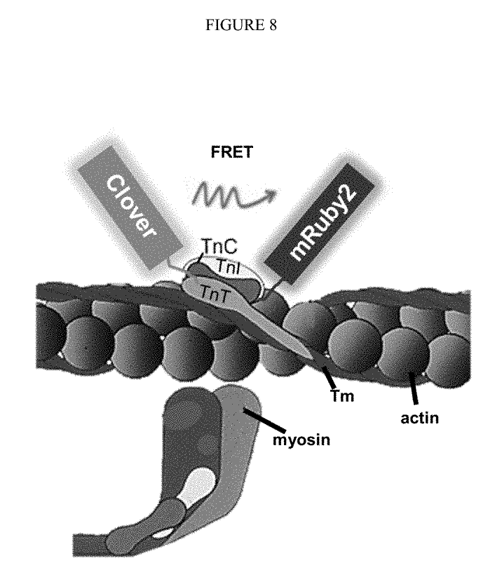

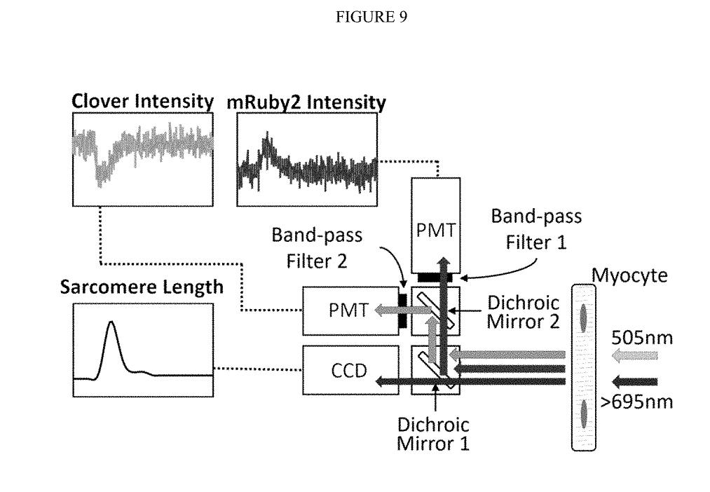

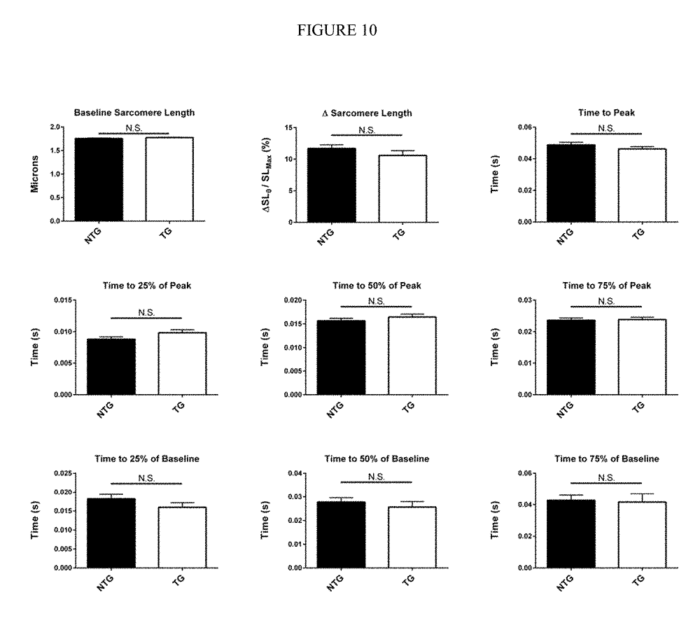

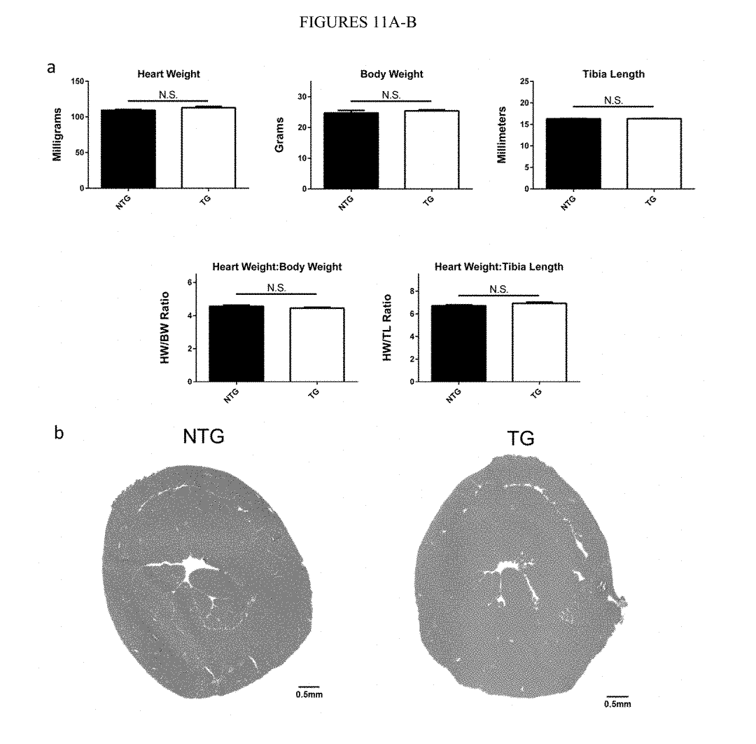

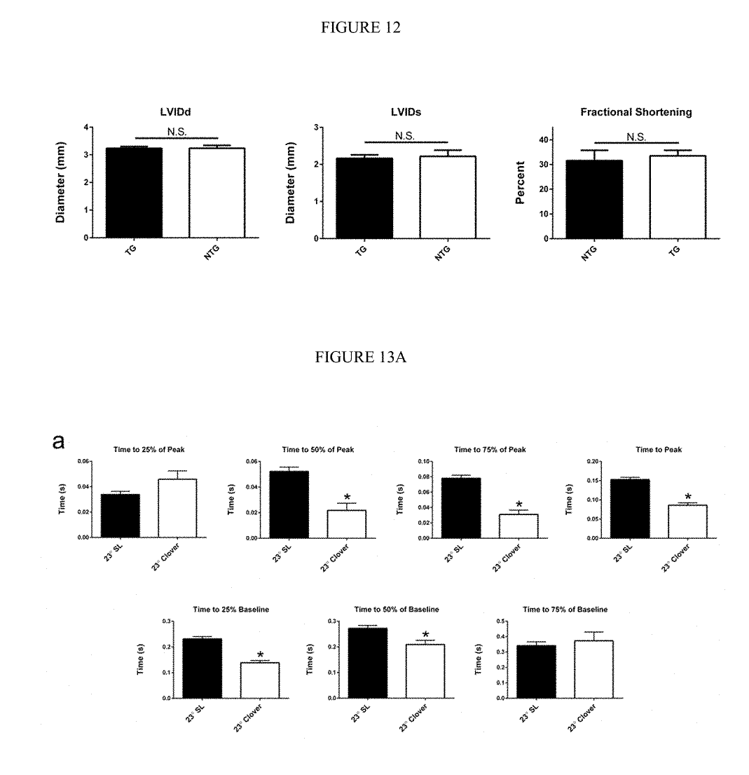

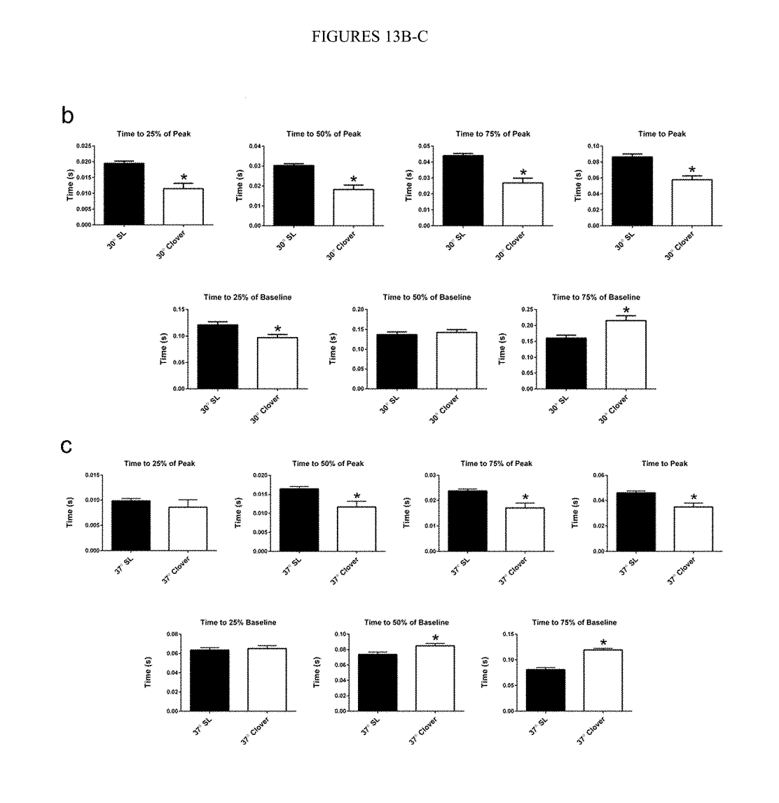

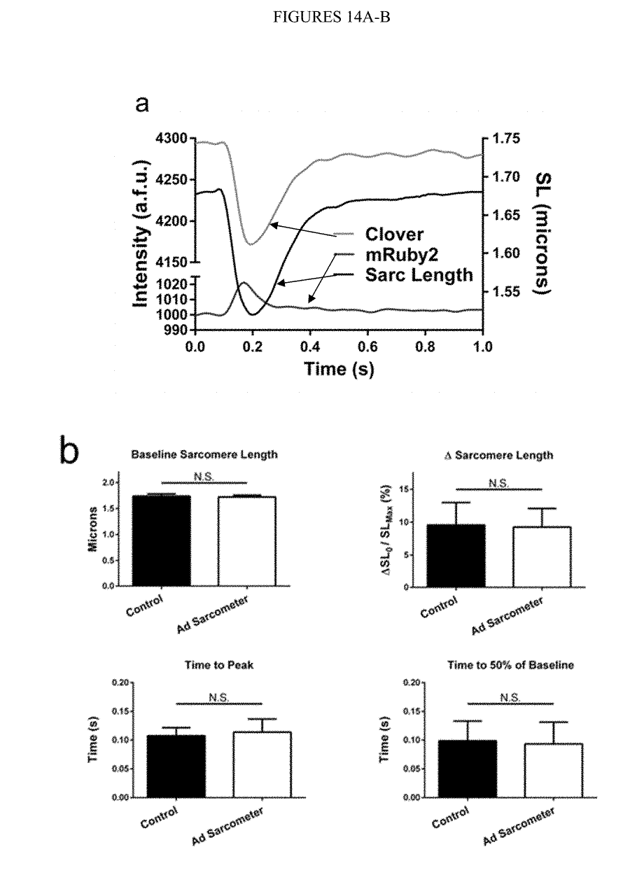

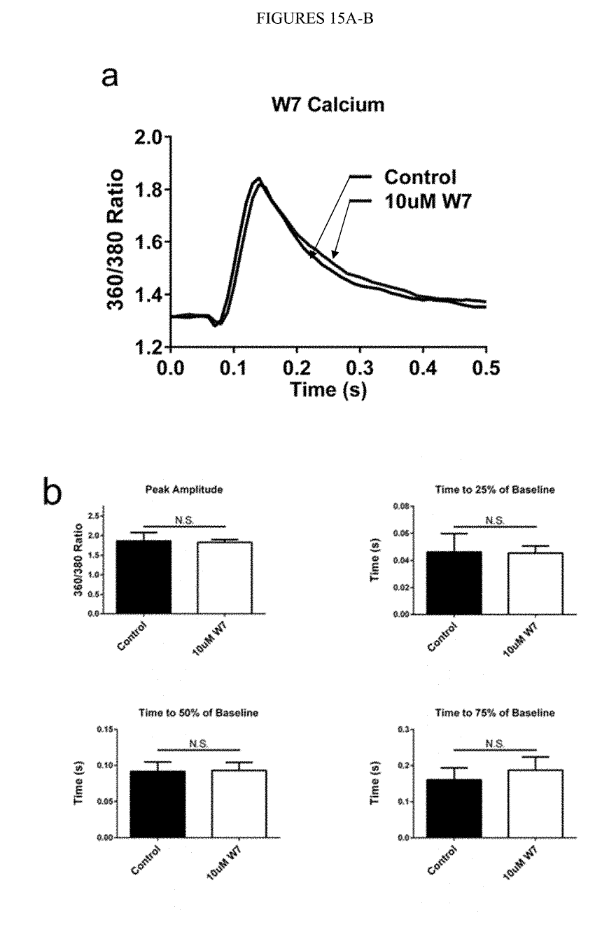

Described herein is the development and implementation of an approach enabling the first investigation of sarcomeric activating ligands in live cardiac myocytes. A sarcomere activation biosensor (herein, Sarcometer) was designed and validated using flexible linkers fusing the fluorescent proteins Clover (donor) and mRuby2 (acceptor) engineered at the N- and C-terminal ends of cardiac TnC, respectively (Methods; FIG. 8, 9). TnC is ideally positioned to serve as a sarcomere activation biosensor as both near and long-range myofilament signaling by Ca²⁺, TnI, Tm and myosin during contraction have been shown to transduce conformational changes in TnC and is detectable by fluorescence resonance energy transfer (FRET), at least when studied under steady-state in vitro conditions (Gordon, et al. (2000) Physiol Rev. 80, 853-924; Hannon, et al. (1992) Circ. Res. 71, 984-991; Zot, A. S. & Potter, J. D. (1989) Biochemistry 28, 6751-6756; Dong, et al. (1999) J. Biol. Chem. 274, 31382-31390). Recent biochemical studies using purified troponin complex with similar FRET pairs reported Ca²⁺ affinity values comparable to those of wild-type troponin (Badr, et al. (2016) PLoS. One. 11, e0164222). Importantly, as inherited mutations in the sarcomere can directly cause cardiomyopathy (Seidman, J. G. & Seidman, C. (2001) Cell 104, 557-567), we validated that the Sarcometer itself had no significant effects on heart histology or morphology, or on intact cardiac myocyte function or heart performance in vivo, as determined in Sarcometer transgenic mice (FIG. 10-13c). Data show that in intact adult cardiac myocytes, the Sarcometer Clover and mRuby2 transients were anti-correlated, indicating an intramolecular conformational change signifying FRET (FIG. 4d,e, FIG. 9). Based on this, together with previous work in reconstituted systems (Dong, et al. (1999) J. Biol. Chem. 274, 31382-31390; Badr, et al. (2016) PLoS. One. 11, e0164222) (and results below), we interpret the Sarcometer FRET signal as reporting in real-time the regulated activation status of the sarcomere in live cardiac myocytes. In independent Sarcometer Tg mouse lines, the Sarcometer stoichiometrically replaced endogenous cTnC (FIG. 4b), with immunofluorescence demonstrating correct localization within the myofilaments of intact myocytes (FIG. 4c). Similar findings were obtained in rat adult cardiac myocytes using acute gene transfer (FIG. 14).

Whereas monitoring cardiac muscle electrical properties and intracellular Ca′ signaling has been possible for decades (Bers, D. M. (2002) Nature 415, 198-205), we sought to use the Sarcometer to investigate dynamic sarcomere activation in live adult cardiac myocytes at 37° C. that had only been estimated from modeling studies previously (Robertson, et al. (1981) Biophys. J. 34, 559-569). Results show the Sarcometer signal initiated earlier and reached peak amplitude prior to Sarcomere Length (SL), a result at apparent odds with present theory that strong myosin binding is requisite to advance the myofilament activation to the open state where active force could be produced (FIG. 4d-e, FIG. 13a-c). Moreover, the initial phase of decay in the Sarcometer signal preceded the early phase of SL relaxation (FIG. 4d-e, FIG. 13a-c). Also, the Clover transient was temperature sensitive, paralleling the temperature dependence of contraction (FIG. 13a-c) consistent with the temperature dependence of the troponin-Tm regulatory system (Gordon, et al. (2000) Physiol Rev. 80, 853-924). Together these findings give support to the view that Ca′ binding to TnC and/or TnI binding to TnC are sufficient as regulatory activating ligands of the sarcomere.

Ca²⁺ and Troponin I Function as Requisite Ligands of Activation in Live Cardiac Myocytes.

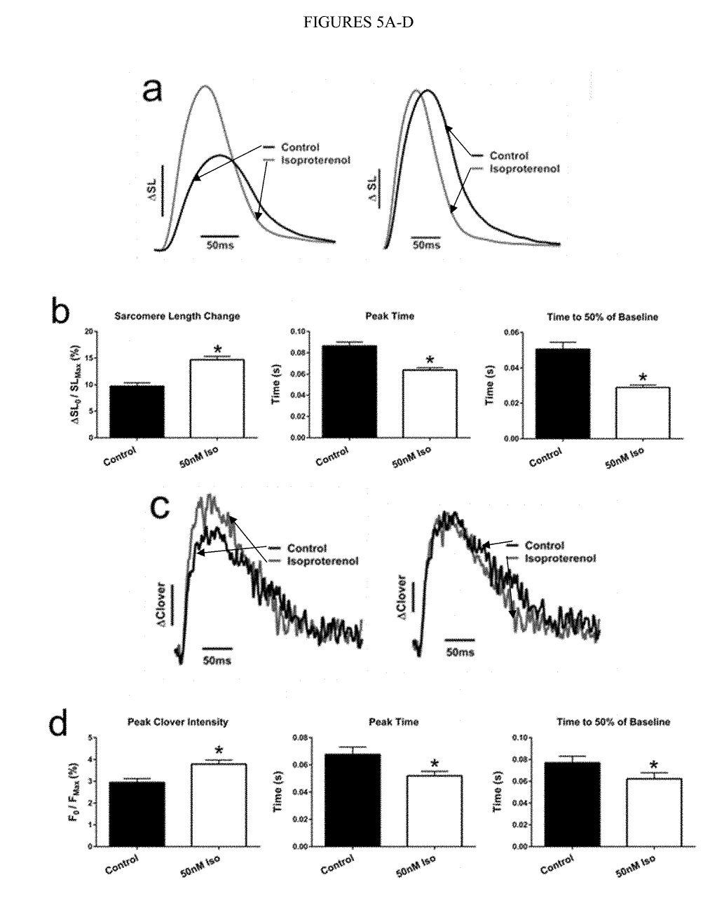

Using the Sarcometer, the dynamics of enhanced intact cardiac myocyte excitation-contraction coupling and sarcomere regulation during β-adrenergic stimulation was probed (Isoproterenol) (Bers, D. M. (2002) Nature 415, 198-205). Here, the Sarcometer Clover peak amplitude was increased and the Clover transient decay rate was significantly faster with isoproterenol, both qualitatively similar to the effects of isoproterenol to increase myocyte contraction (positive inotropy) and to speed SL relaxation (positive lusitropy) (FIG. 5a-d). The faster inactivation of the Clover signal by isoproterenol was interpreted as consistent with the faster myofilament inactivation predicted due to cTnI phosphorylation, along with faster Ca²⁺ transient decay (Yasuda, et al. (2007) Circ. Res. 101, 377-386).

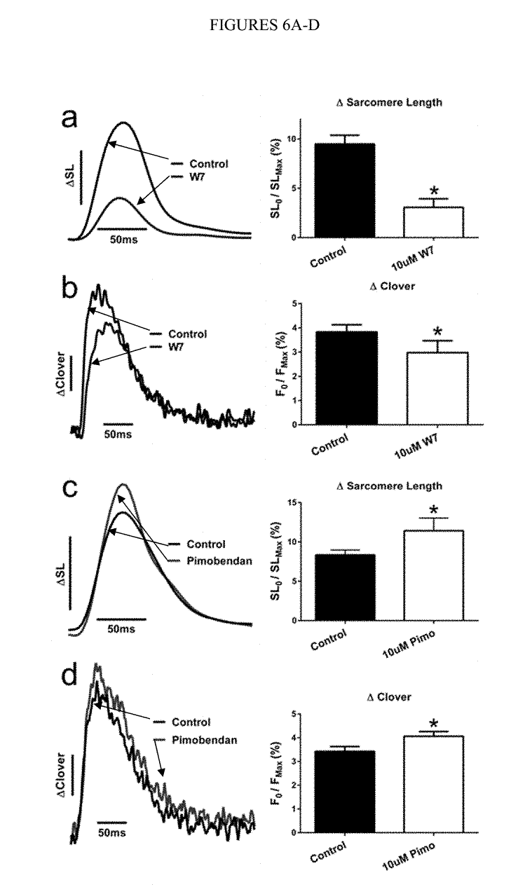

To more precisely interrogate live cell sarcomere activation cTnI was investigated as a required activating ligand. Biochemical evidence suggests that the cTnI switch peptide binding to cTnC is required for the full activation of cTnC, unlike in skeletal muscle where Ca′ binding alone is sufficient for full TnC activation (Sykes, B. D. (2003) Nat. Struct. Biol. 10, 588-589; Sia, et al. (1997) J. Biol. Chem. 272, 18216-18221). To pursue this line of investigation we first used N-aminohexyl-5-chloro-1-naphthalenesulfonamide (W7), a small molecule demonstrated to bind directly to the TnC hydrophobic patch to decrease TnI switch peptide binding by 13-fold (Oleszczuk, et al. (2010) J. Mol. Cell Cardiol. 48, 925-933), and used here to test the effects of disrupted TnC-TnI interaction on sarcomere activation in live myocytes. Here, the Clover amplitude was significantly depressed by W7 (FIG. 6b), paralleling the reduced SL amplitude by W7 (FIG. 6a). Because the Ca²⁺ transient was unaltered by W7 (FIG. 15a-b) the Sarcometer is not monitoring myoplasmic Ca²⁺. Thus, in conjunction with the switch-peptide occlusion mechanism (Sykes, B. D. (2003) Nat. Struct. Biol. 10, 588-589; Sia, et al. (1997) J. Biol. Chem. 272, 18216-18221) and the effects of W7 on contraction (Thompson, et al. 2016) Am. J. Physiol Heart Circ. Physiol ajpheart), these data are taken as evidence that the Sarcometer directly tracks sarcomere activation and establishes the key role of TnC-TnI interaction in live cells by reporting the regulatory alterations in myofilament structural conformations (Gordon, et al. (2000) Physiol Rev. 80, 853-924; Sykes, B. D. (2003) Nat. Struct. Biol. 10, 588-589; Tobacman, L. S. (1996) Annu. Rev. Physiol 58, 447-481).

Whether enhancing TnC-TnI regulatory function would be detected in live cells via the Sarcometer was also tested. In cardiac myocytes exposed to Pimobendan, a positive inotrope (Hwang, P. M. & Sykes, B. D. (2015) Nat. Rev. Drug Discov. 14, 313-328) that directly enhances the affinity of cTnC for cTnI (Schlecht, et al. (2016) Chem. Biol. Drug Des 87, 171-181), there was a significant increase in Clover signal amplitude that was qualitatively similar to the increased SL amplitude (FIG. 6c,d). Thus the Sarcometer reports the enhanced activation of the myofilament regulatory system elicited by increasing the affinity of cTnI for cTnC's hydrophobic cleft. Collectively, these findings by titrating cTnC-cTnI interactions, either increased or decreased, demonstrate that cTnI engagement with cTnC is a key effector of cardiac sarcomere activation in the live cell.

Myosin Binding is not an Essential Regulator of Myofilament Activation.

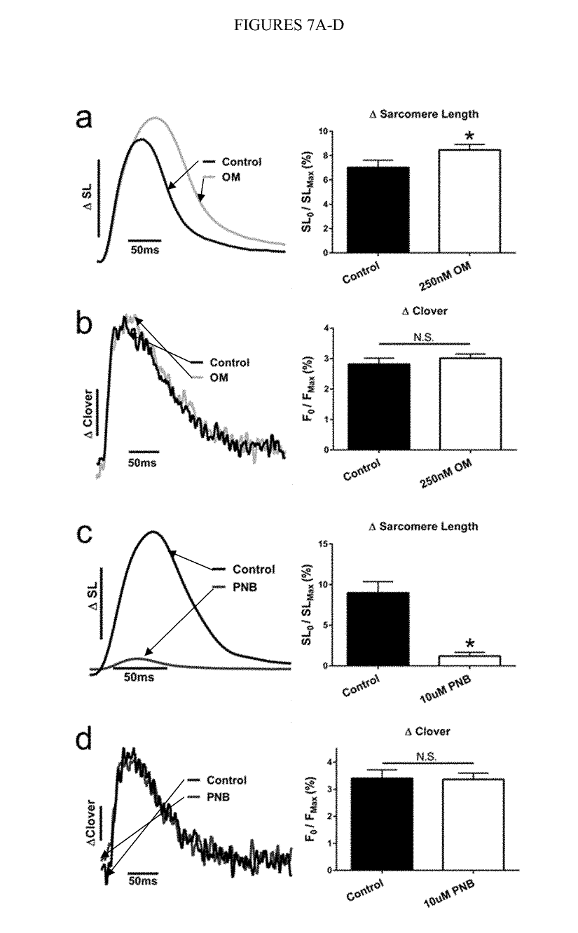

Strong myosin binding has long been proposed to be the essential ligand required for the open state of the myofilament regulatory system (Gordon, et al. (2000) Physiol Rev. 80, 853-924; Bremel, R. D. & Weber, A. (1972) Nat. New Biol. 238, 97-101; McKillop, D. F. & Geeves, M. A. (1993) Biophys. J. 65, 693-701). To test the myosin binding-based theory of sarcomere activation in live cardiac myocytes Omecamtiv mecarbil (OM) was first used, a myosin activator known to increase the rate of cross-bridge cycling by accelerating phosphate release (Malik, et al. (2011) Science 331, 1439-1443). As expected, OM caused a significant increase SL amplitude in Tg adult cardiac myocytes but, surprisingly, OM had no effect on Sarcometer amplitude or kinetics (FIG. 7a,b). This result was unexpected given the extensive literature on strong myosin binding as the required allosteric effector of sarcomere activation (Gordon, et al. (2000) Physiol Rev. 80, 853-924; McKillop, D. F. & Geeves, M. A. (1993) Biophys. J. 65, 693-701; Geeves, M. A. & Holmes, K. C. (2005) Adv. Protein Chem. 71, 161-193; Geeves, M. A. & Lehrer, S. S. (2014) Biophys. J. 107, 543-545). To more thoroughly test this hypothesis, it was investigated whether fully blocking strong myosin binding would depress the Sarcometer signal, as predicted (Gordon, et al. (2000) Physiol Rev. 80, 853-924; McKillop, D. F. & Geeves, M. A. (1993) Biophys. J. 65, 693-701; Geeves, M. A. & Holmes, K. C. (2005) Adv. Protein Chem. 71, 161-193; Geeves, M. A. & Lehrer, S. S. (2014) Biophys. J. 107, 543-545). Here, Sarcometer Tg cardiac myocytes were exposed to para-Nitroblebbistatin (PNB), a potent small molecule that selectively disables myosin function by blocking myosin in an actin-detached state and therefore incapable of producing force (Kepiro, et al. (2014) Angew. Chem. Int. Ed Engl. 53, 8211-8215). Most dramatically, wherein PNB effectively blocked contraction (FIG. 7c), there was no significant effect of the myosin-based inhibited contraction on either Sarcometer amplitude or kinetics (FIG. 7d). These findings are new evidence in live cells under physiological dynamic Ca²⁺ handling that strong cross-bridge binding is not essential for regulating cardiac sarcomere activation.

### Discussion

Reported herein are the first studies, to the inventor's knowledge, on the regulatory mechanisms of live cell cardiac sarcomere activation. Evidence indicates that activating ligands Ca²⁺ and TnI are sufficient as allosteric effectors to activate the myofilaments in live adult cardiac myocytes. Furthermore, in contradistinction to long-standing theory, the data show that strong myosin binding is not necessary to activate the sarcomeric regulatory system in the dynamic physiological setting of the live cardiac myocyte. It is speculated that previous myosin binding-based activation studies (Gordon, et al. (2000) Physiol Rev. 80, 853-924; Geeves, M. A. & Holmes, K. C. (2005) Adv. Protein Chem. 71, 161-193; Metzger, J. M. (1995) Biophys. J. 68, 1430-1442) are valid for the steady-state conditions tested but, as shown here, do not extend to the dynamic conditions of intact physiological excitation-contraction (EC) coupling of the live cell under the conditions tested here.

Taken together, these findings may have significant implications for understanding the fundamental mechanisms that govern beat-to-beat control of cardiac contraction. For example, in several inherited and acquired cardiac diseases, sarcomeric dysfunction, not Ca²⁺ mishandling, underlies disease initiation and this is termed EC uncoupling (Seidman, J. G. & Seidman, C. (2001) Cell 104, 557-567; Lee, J. A. & Allen, D. G. (1991)J. Clin. Invest 88, 361-367; Davis, et al. (2012) J Mol. Cell Cardiol. 53, 446-457). Here, disease severity couples closely with the degree of sarcomere-based EC-uncoupling. In acute ischemia, hypoxia, and acidosis, the intracellular Ca²⁺ transient is normal and yet sarcomere-based heart pump performance is severely depressed (Lee, J. A. & Allen, D. G. (1991)J. Clin. Invest 88, 361-367). Furthermore, in inherited cardiomyopathies premature sudden cardiac death can result from sarcomeric mutations uncoupled from Ca²⁺ transient disturbances (Seidman, J. G. & Seidman, C. (2001) Cell 104, 557-567). These are evidence of the critical role of sarcomere performance in cardiac health and disease.

Advances here may now permit further detailed insights into the function of the sarcomere in health and disease, including potentially elucidating the roles of new sarcomeric activating ligands (Hwang, P. M. & Sykes, B. D. (2015) Nat. Rev. Drug Discov. 14, 313-328) and post-translation myofilament modifications (Yasuda, et al. (2007) Circ. Res. 101, 377-386) that, heretofore, have not been possible to test in live myocytes. This study may also enable future investigations of sarcomere regulation under varying physiological conditions including pacing, stress, load, and across species. Insights into disease mutations, new drugs and small molecules that specifically target sarcomere activation may also be forthcoming. Finally, it would be interesting to develop and test a Sarcometer for live skeletal muscle study. It is speculated, owing to the significant differing mechanism of troponin-based sarcomere regulation in heart versus skeletal muscle (Sia, et al. (1997) J. Biol. Chem. 272, 18216-18221), that ligands of activation may fundamentally differ between the striated muscle lineages when studied in live cells.

### Material and Methods

### Animal Work

The procedures used in this study were approved under guidelines of the University of Minnesota Committee on the Use and Care of Animals. Adult ventricular cardiac myocytes were isolated from adult female Sprague Dawley rats or adult C57/Bl6 mice of both sexes.

### Gene Construct, Recombinant Vectors, and Transgenesis

The 480 bp full length (less then starting methionine) human cardiac Troponin C (hcTnC) cDNA was synthesized de novo and engineered to be flanked on the 5′ end by a 20 amino acid flexible linker peptide enriched in leucine, glycine, serine, and alanine; and flanked on the 3′ end by an 8 amino acid flexible linker enriched in leucine and alanine. The 20 amino acid linker is inserted between a PasI restriction site at the 5′ end and an EcoRV restriction site at the 3′ end. The 8 amino acid linker is inserted between a PfoI restriction site at the 5′ end and a BclI restriction site at the 3′ end. On the 5′ end of the 20 amino acid flexible linker is the 684 bp cDNA of the green fluorescent protein “Clover”. The Clover cDNA is inserted between a SalI restriction site at the 5′ end and the aforementioned PasI restriction site at the 3′ end. On the 3′ end of the 8 amino acid flexible linker is the 714 bp cDNA of the red fluorescent protein “mRuby2”. The mRuby2 cDNA is inserted between the aforementioned MI restriction site at the 5′ end and an FseI restriction site at the 3′ end. The Sarcometer gene construct was inserted in a DNA vector downstream of an alpha myosin heavy chain (aMHC) promoter element for robust and cardiac specific expression in a mammalian system. This aMHC Sarcometer vector was inserted by pronuclear injection into donor egg cells and implanted in C57/B16 murine surrogate mothers. Live pups were genotyped using the PCR sense primer located in mRuby2: 5′-CAACATCAAGATGCCCGGCA-3′, and the PCR antisense primer located in the SV40 PolyA region: 5′-CACTGCATTCTAGTTGTGGTTTGTCC-3′ with an annealing temperature of 62° C. Of the live pups, 5 founders carrying the transgene were identified and the line with greatest breeding vigor was selected for further validation.

For recombinant adenovirus, a similar DNA construct was synthesized de novo but containing HindIII and EcoRI at the 5′ and 3′ ends respectively for insertion into a pDC316 recombinant adenovirus production vector. pDC316-Sarcometer and an adenovirus genome cosmid were co-transfected by calcium phosphate into confluent HEK293 cells. Noble agar was overlaid after transfection and clonal viral plaques were amplified. High-titer plaque-purified stocks were produced and viral aliquots were stored at −80° C. (Westfall, et al. (1997) Methods Cell Biol. 52, 307-322).

### Heart Morphology and In Vivo Echocardiography

After initial weigh-in, eight to twenty week old NTG and TG mice of both sexes were euthanized by sodium pentobarbital injection. Hearts were excised and rinsed of excess blood and weighed. Hindlimbs were degloved and the tibia was exposed by blunt dissection prior to measurement.

For hematoxylin and eosin staining, fresh frozen slides were fixed with Bouin's reagent then washed in 70% ethanol followed by water. They were then briefly stained with hematoxylin and washed in tap water. Before eosin staining, slides were dipped briefly in Scott's Tap Water (Sigma cat#55134) and then counterstained with eosin. After rinsing eosin stain, slides were dehydrated with ethanol and then Formula 83 (CBG Biotech cat#CH0104).

Eight to twenty week old Ntg and Sarcometer Tg mice of both sexes were lightly anesthetized by 2.5% Isoflurane and prepared for echocardiography. M-mode imaging was performed with a Vevo 2100 (Visual Sonics) at 1000 fps with the MS550D 22-55 MHz transducer. The parasternal short axis view was used to assess systolic and diastolic ventricle size. M-mode imaging was also used to calculate fractional shortening.

### Myocyte Isolation and Culture

Eight to twelve week old mice of both sexes were injected with 0.25 cc 10,000U/ml heparin and anesthetized 10 minutes later with 0.15 cc 50 mg/ml sodium pentobarbital. The heart was rapidly excised and placed in ice cold Krebs-Henseleit buffer modified with 25 mM HEPES, 10 mM 2,3-Butanedione monoxime, 30 mM Taurine and the aorta was cannulated. The mounted heart was placed on a perfusion system at 37° C. The heart was perfused with calcium free modified Krebs-Henseleit buffer with 2 mg/ml crudely purified collagenase for 10 minutes. The heart was then removed from the cannula, the atria were removed and the ventricles were dissected into pieces approximately 2 mm in diameter. The dissected ventricular tissue was then gently mechanically triturated in collagenase to liberate isolated myocytes. After brief low-speed centrifugation, isolated myocytes were resuspended in nominal calcium and 0.025 mg/ml bovine serum albumin in a modified Kreb-Henseleit buffer. After the final trituration step, calcium was added back to the buffer step wise until reaching a final concentration of 2 mM CaCl₂. Myocytes were plated on laminin-coated (ThermoFisher cat#23017015) glass coverslips in M199 media (Gibco cat#31100-035) containing 5% fetal bovine serum and incubated for one hour at 37° C. with 5% CO₂. After the one hour incubation, the media was changed to serum free M199 media and the myocytes were used for experiments within 5 hours.

### Real-Time Calcium, Contractility, and Sarcomere Biosensor Measurements

For real-time simultaneous sarcomere length and Sarcometer biosensor measurements, a modified Ionoptix Calcium and Contractility system was used. Sarcomere length changes were detected at 240 Hz sampling rate and fluorescence measurements were detected at 1000 Hz sampling rate with two photomultipler tubes (FIG. 9). Measurements were made at 37° C. and a stimulation frequency of 0.2 Hz unless specified otherwise. For Sarcometer measurements, a Xenon arc lamp was used and filtered with a 500/10 nm hard-coated bandpass filter before entering the microscope turret. The microscope turret featured a 505 dichroic long pass filter. Fluorescent light from the sample was shunted through microscope side port where it was split by a dichroic mirror with light above 650 nm going toward the Myocam for sarcomere length detection and light below 650 nm being further shunted to an additional filter cube. In this final filter cube, a 565 nm dichroic long pass mirror split the light again. Light with wavelengths below 565 nm corresponding to the Clover protein was further cleaned up by a 534/20 nm hard-coated bandpass filter before reaching the photomultiplier tuber and light with wavelengths above 565 nm was further cleaned up by a 592/43 nm hard-coated bandpass filter before reaching the photomultiplier tube. For calcium fluorescence measurements, the mouse myocytes were pre-loaded for 20 minutes in M199 solution containing 1 μM Fura-2 AM (ThermoFisher Cat #F1221), and a ten minute period was allowed for deesterification. For calcium fluorescence measurements the 360/380 nm ratio was determined. For each myocyte, several contractions were measured, averaged, and analyzed using the IonWizard software.

For adenovirus transduced biosensor experiments, adult cardiac myocytes were isolated as previously described (Geeves, M. A. & Lehrer, S. S. (2014) Biophys. J. 107, 543-545). Briefly, the myocytes were isolated by collagenase digestion and mechanical trituration prior to plating on laminin coated coverslips. Myocytes were treated with AdSarcometer at a multiplicity of infection of 100 for 48 hrs prior to measurement with the aforementioned IonOptix calcium and contractility system.

### Western Blotting and Immunofluorescence

The primary antibodies used were to α-actinin (immunofluorescence) (1:500, Abcam, EA-53), and cardiac troponin C (western blot) (1:2500, Novus Biologicals, 1A2). The secondary antibodies were conjugated to Alexa Fluor 647 (ThermoFisher) and IRDye Alexa Fluor 680 (ThermoFisher) respectively. For Western blots signals were visualized and quantified using the Odyssey system (Licor). Laminin coated coverslips containing isolated myocytes were treated with 4% paraformaldehyde in phosphate buffered saline for five minutes followed by two washes in phosphate buffered saline. Samples were blocked with 5% bovine serum albumin with 0.5% Triton X-100 in phosphate buffered saline for at least one hour. Primary antibody was applied at room temperature for one hour in phosphate buffered saline with 0.5% Triton X-100 and 0.5% BSA. Several washes were performed in PBS with 0.5% Triton X-100 and 5% BSA, followed by one hour incubation of the secondary antibody at room temperature in PBS with 0.5 Triton X-100 and 0.5% BSA. Coverslips were mounted on microscope slides with ProLong Diamond mounting medium. Myocytes were imaged using a Nikon A1R confocal microscope with 60× water-immersion objective; myocytes were illuminated sequentially in line scanning mode by 488 nm, 561 nm, and 640 nm lasers.

### Statistics

For contractility and calcium analysis, curve fitting was performed on unfiltered, averaged single myocyte transients (IonWizard 6.5; IonOptix). Mean±S.E.M. are presented in histogram summary figures. For isoproterenol, W7, pimobendan, omecamtiv mecarbil, and para-Nitroblebbistatin studies two-tailed paired t-tests were performed. For all other summary histograms, two-tailed unpaired t-tests were performed (GraphPad PRISM version 6.0; GraphPad Software Inc.). Sample sizes were chosen on basis of Power analysis informed by previous experience in the laboratory with respect to biological variability. P values<0.05 were considered statistically significant, and indicated by asterisks in figures.

All publications, patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

What is claimed is:

1. A polypeptide comprising:
(a) an amino acid sequence encoding a first chromophore;
(b) a first linker;
(c) an amino acid sequence having at least about 80% sequence identity to an amino acid sequence encoding a troponin C;
(d) a second linker; and
(e) an amino acid sequence encoding a second chromophore;
wherein the first and second chromophores are a Förster resonance energy transfer (FRET) donor-acceptor pair; wherein the first chromophore is a donor chromophore and the second chromophore is an acceptor chromophore, or wherein the first chromophore is an acceptor chromophore and the second chromophore is a donor chromophore; and wherein the polypeptide is capable of integrating into a sarcomere.

2. The polypeptide of claim 1, wherein the first and/or second chromophore is a fluorophore selected from the group consisting of Aquamarine, mCerulean, mTurquoise, mTurquoise2, CyPet, SCFP3A, Amber, mVenus, Ypet, SYFP2, SYFP2A, Clover, LSSmOrgange, mRuby2, ECFP, CFP, YFP, GFP, EGFP, Citrine, EYFP, mCherry and DsRed.

3. The polypeptide of claim 2, wherein the first or second chromophore is Clover and has at least about 80% sequence identity to SEQ ID NO:2.

4. The polypeptide of claim 2, wherein the first or second chromophore is mRuby2 and has at least about 80% sequence identity to SEQ ID NO:3.

5. The polypeptide of claim 1, wherein the first linker is about 18 to about 22 amino acids in length.

6. The polypeptide of claim 1, wherein the first linker is enriched in leucine, glycine, serine, and/or alanine residues.

7. The polypeptide of claim 1, wherein the first linker comprises an amino acid sequence having about 85% sequence identity to LGGGGSGGGGSGGGGSAAAG (SEQ ID NO:4).

8. The polypeptide of claim 1, wherein the second linker is about 6 to about 10 amino acids in length.

9. The polypeptide of claim 1, wherein the second linker is enriched in leucine and/or alanine residues.

10. The polypeptide of claim 1, wherein the second linker comprises an amino acid sequence having about 85% sequence identity to LAAALAAA (SEQ ID NO:5).

11. The polypeptide of claim 1, wherein the troponin C is human TNNC1 and has at least about 80% sequence identity to SEQ ID NO:6.

12. The polypeptide of claim 1, comprising in order:
(a) an amino acid sequence encoding Clover;
(b) a first linker;
(c) an amino acid sequence encoding human TNNC1;
(d) a second linker; and
(e) an amino acid sequence encoding mRuby2;
wherein the polypeptide is capable of integrating into a sarcomere.

13. The polypeptide of claim 12, wherein the polypeptide comprises an amino acid sequence having at least about 80% sequence identity to SEQ ID NO:1.

14. A nucleic acid encoding a polypeptide of claim 1.

15. A vector comprising an expression cassette comprising a promoter and the nucleic acid of claim 14, wherein the promoter is operably linked to the nucleic acid.

16. A cell comprising the vector of claim 15.

17. A transgenic non-human animal whose genome comprises a DNA transgene, which encodes a polypeptide of claim 1.

18. A method for detecting structural changes in troponin C or sarcomere activation in a living cell, comprising 1) measuring in a FRET assay the fluorescent emission from a living cell expressing a polypeptide as described in claim 1; and 2) detecting structural changes in troponin C or sarcomere activation when changes in the fluorescent emission are detected.

19. A method for screening biologically active agents that inhibit or promote sarcomere activation, comprising 1) contacting a living cell expressing a polypeptide as described in claim 1 with a test biologically active agent; 2) measuring the fluorescent emission from the cell in a FRET assay; and 3) detecting an effect of the test agent on sarcomere activation and thereby identifying an agent that inhibits or promotes sarcomere activation.

20. A method for screening sarcomere protein mutations that inhibit or promote sarcomere activation, comprising 1) measuring the fluorescent emission from a living cell in a FRET assay, wherein the living cell expresses a polypeptide as described in claim 1 and comprises one or more mutations in a sarcomere protein; and 2) detecting an effect of the one or more mutations on sarcomere activation and thereby identifying a mutation that inhibits or promotes sarcomere activation.

21. The method of claim 20, wherein the sarcomere protein is Telethonin, Capz, Titin, Myopalladin, Nebulin, Nebulette, cTnI, fsTnI, ssTnI, cTnC/ssTnC, fsTnC, cTnT, fsTnT, ssTnT, fsMyBP-C, ssMyBP-C, cMyBP-C, alpha Myosin Heavy Chain, beta Myosin Heavy Chain, Myosin Heavy Chain 7B, MYL1 (fast skeletal LC), MYL2 (ventricular RLC), MYL3 (ventricular ELC), MYL4 (atrial ELC), MYL5 (fetal RLC), MYL6B (slow skeletal LC), MYL7 (atrial RLC), MYL11 (fs phosphorylatable LC), skeletal Actin, cardiac Actin, alpha Actinin 2, Obscurin, Ankyrin, Tropomyosin 1, Tropomyosin 2, Tropomyosin 3 or Tropomyosin 4.


SEQUENCE LISTING (11 sequences)

SEQ ID NO: 1 - protein, 653 aa, Artificial Sequence
  Description of Artificial Sequence Synthetic polypeptide
MVSKGEELFTGVVPILVELDGDVNGHKFSVRGEGEGDATNGKLTLKFICTTGKLPVPWPTLVTTFGYGVACFSRYPDHMKQHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSHQSALSKDPNEKRDHMVLLEFVTAALGGGGSGGGGSGGGGSAAAGDDIYKAAVEQLTEEQKNEFKAAFDIFVLGAEDGCISTKELGKVMRMLGQNPTPEELQEMIDEVDEDGSGTVDFDEFLVMMVRCMKDDSKGKSEEELSDLFRMFDKNADGYIDLDELKIMLQATGETITEDDIEELMKDGDKNNDGRIDYDEFLEFMKGVELAAALAAAMVSKGEELIKENMRMKVVMEGSVNGHQFKCTGEGEGNPYMGTQTMRIKVIEGGPLPFAFDILATSFMYGSRTFIKYPKGIPDFFKQSFPEGFTWERVTRYEDGGVVTVMQDTSLEDGCLVYHVQVRGVNFPSNGPVMQKKTKGWEPNTEMMYPADGGLRGYTHMALKVDGGGHLSCSFVTTYRSKKTVGNIKMPGIHAVDHRLERLEESDNEMFVVQREHAVAKFAGLGGGMDELYK

SEQ ID NO: 2 - protein, 228 aa, Artificial Sequence
  Description of Artificial Sequence Synthetic polypeptide
MVSKGEELFTGVVPILVELDGDVNGHKFSVRGEGEGDATNGKLTLKFICTTGKLPVPWPTLVTTFGYGVACFSRYPDHMKQHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSHQSALSKDPNEKRDHMVLLEFVTAA

SEQ ID NO: 3 - protein, 237 aa, Artificial Sequence
  Description of Artificial Sequence Synthetic polypeptide
MVSKGEELIKENMRMKVVMEGSVNGHQFKCTGEGEGNPYMGTQTMRIKVIEGGPLPFAFDILATSFMYGSRTFIKYPKGIPDFFKQSFPEGFTWERVTRYEDGGVVTVMQDTSLEDGCLVYHVQVRGVNFPSNGPVMQKKTKGWEPNTEMMYPADGGLRGYTHMALKVDGGGHLSCSFVTTYRSKKTVGNIKMPGIHAVDHRLERLEESDNEMFVVQREHAVAKFAGLGGGMDELYK

SEQ ID NO: 4 - protein, 20 aa, Artificial Sequence
  Description of Artificial Sequence Synthetic peptide
LGGGGSGGGGSGGGGSAAAG

SEQ ID NO: 5 - protein, 8 aa, Artificial Sequence
  Description of Artificial Sequence Synthetic peptide
LAAALAAA

SEQ ID NO: 6 - protein, 160 aa, Homo sapiens
DDIYKAAVEQLTEEQKNEFKAAFDIFVLGAEDGCISTKELGKVMRMLGQNPTPEELQEMIDEVDEDGSGTVDFDEFLVMMVRCMKDDSKGKSEEELSDLFRMFDKNADGYIDLDELKIMLQATGETITEDDIEELMKDGDKNNDGRIDYDEFLEFMKGVE

SEQ ID NO: 7 - DNA, 20 bp, Artificial Sequence
  Description of Artificial Sequence Synthetic primer
caacatcaagatgcccggca

SEQ ID NO: 8 - DNA, 26 bp, Artificial Sequence
  Description of Artificial Sequence Synthetic primer
cactgcattctagttgtggtttgtcc

SEQ ID NO: 9 - DNA, 1962 bp, Artificial Sequence
  Description of Artificial Sequence Synthetic polynucleotide
atggtgtccaagggcgaggaactgttcaccggcgtggtgcccatcctggtggaactggatggcgacgtgaacggccacaagttctctgtgcggggagagggcgaaggcgacgccacaaacggaaagctgaccctgaagttcatctgcaccaccggcaagctgcccgtgccttggcctaccctcgtgaccacatttggctacggcgtggcctgcttcagcagataccccgaccatatgaagcagcacgacttcttcaagagcgccatgcccgagggctacgtgcaggaaagaaccatcagcttcaaggacgacggcacctacaagaccagagccgaagtgaagttcgagggcgacaccctcgtgaacagaatcgagctgaagggcatcgacttcaaagaggacggcaacatcctgggccacaagctggagtacaacttcaacagccacaacgtgtacatcaccgccgacaagcagaagaacggcatcaaggccaacttcaagatcaggcacaacgtggaagatggcagcgtgcagctggccgaccactaccagcagaacacccctatcggcgacggccctgtgctgctgcctgacaaccactacctgagccaccagagcgccctgagcaaggaccccaacgagaagagggaccacatggtgctgctggaatttgtgacagccgccctgggcggaggcggatctggcggcggaggatcagggggagggggatctgctgccgccggagacgatatctacaaggccgccgtggaacagctgaccgaggaacagaagaatgagttcaaggccgctttcgacatcttcgtgctgggcgccgaggacggctgcatcagcacaaaagaactgggcaaagtgatgaggatgctgggccagaaccccacccctgaggaactgcaggaaatgatcgacgaggtggacgaggacggaagcggcaccgtggacttcgacgagttcctcgtgatgatggtgcgctgcatgaaggacgacagcaagggcaagagcgaggaagaactgagcgacctgttcaggatgttcgacaagaacgccgacggctacatcgacctggatgagctgaagatcatgctgcaggccacaggcgagacaatcaccgaggacgacatcgaggaactgatgaaggacggcgacaagaacaacgatggcagaatcgactacgatgagttcctggagttcatgaagggcgtggaactggccgctgccctggctgcagctatggtgtctaaaggggaagaactgatcaaagaaaacatgcggatgaaggtcgtgatggaaggcagcgtgaacggacatcagttcaagtgcaccggcgagggcgagggcaacccttacatgggcacccagaccatgagaatcaaagtgatcgaaggcggccctctgcccttcgccttcgacatcctggctaccagctttatgtacggctcccgcaccttcatcaagtaccccaagggcatccccgatttcttcaagcagagcttccctgagggcttcacctgggagagagtgaccagatacgaggatggcggagtcgtgaccgtgatgcaggacaccagcctggaagatggatgcctggtgtaccacgtgcaagtgcggggcgtgaacttccccagcaacggcccagtgatgcagaaaaagaccaagggctgggagcccaacaccgagatgatgtaccctgccgatggcggcctgagaggctacacacacatggccctgaaggtggacggcggaggccacctgagctgtagcttcgtgacaacctacaggtccaagaaaaccgtgggcaacatcaagatgcccggcatccacgccgtggaccacagactggaaaggctggaagagtccgacaacgagatgttcgtggtgcagcgcgagcacgctgtggccaagtttgctggactgggaggcggcatggacgagctgtacaagtga

SEQ ID NO: 10 - DNA, 44 bp, Unknown
  Description of Unknown 3′UTR sequence
acccagctttcttgtacaaagtggtgatggccggccgcttcgag

SEQ ID NO: 11 - DNA, 222 bp, Unknown
  Description of Unknown PolyA_signal sequence
cagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggta